(19) United States
(12) Patent Application Publication
(10) Pub. No.: US 2011/0053884 A1
(43) Pub. Date: Mar. 3, 2011

(54) POTENT COMBINATIONS OF ZIDOVUDINE AND DRUGS THAT SELECT FOR THE K65R MUTATION IN THE HIV POLYMERASE

(76) Inventor: Raymond F. Schinazi, Atlanta, GA (US)

(21) Appl. No.: 12/680,874

(22) PCT Filed: Sep. 29, 2008
(86) PCT No.: PCT/US08/78207

§ 371 Date: Oct. 6, 2010

Related U.S. Application Data
(60) Provisional application No. 60/997,264, filed on Oct. 2, 2007.

(51) Int. Cl. A61K 31/7072; A61P 31/18; A61P 31/14

(52) U.S. Cl. 514/50

(57) ABSTRACT

Combinations of antiretroviral nucleoside reverse transcriptase inhibitors, and methods for their use in treating retroviral infections, are provided. In one embodiment, the combinations include non-thymidine nucleoside antiretroviral agents, such as tenofovir-DF, abacavir, APD and DAPD, that select for the K65R mutation and relatively low doses of zidovudine (AZT) or other thymidine nucleoside antiretroviral agents. The thymidine nucleoside antiretroviral agents retard development of the K65R mutation, and at the low doses, are less likely to produce side effects. In another embodiment, the combinations include DAPD and AZT. DAPD retards the development of TAMs, and AZT retards the development of the K65R mutation. In a third embodiment, the combinations include adenine, cytosine, thymidine, and guanine nucleoside antiviral agents, in further combination with at least one additional antiviral agent that works via a different mechanism than a nucleoside analog. This combination has the potential to eliminate the presence of HIV in an infected patient.

### BACKGROUND OF THE INVENTION

In 1983, the etiological cause of AIDS was determined to be the human immunodeficiency virus (HIV). In 1985, it was reported that the synthetic nucleoside 3′-azido-3′-deoxythymidine (AZT) inhibits the replication of human immunodeficiency virus. Since then, a number of other synthetic nucleosides, including 2′,3′-dideoxyinosine (DDI), 2′,3′-dideoxycytidine (DDC), 2′,3′-dideoxy-2′,3′-didehydrothymidine (D4T), ((1S,4R)-4-[2-amino-6-(cyclopropylamino)-9H-purin-9-yl]-2-cyclopentene-1-methanol sulfate (ABC), cis-2-hydroxymethyl-5-(5-fluorocytosin-1-yl)-1,3-oxathiolane (FTC), (−)-cis-2-hydroxymethyl-5-(cytosin-1-yl)-1,3-oxathiolane (3TC), have been proven to be effective against HIV. After cellular phosphorylation to the 5′-triphosphate by cellular kinases, these synthetic nucleosides are incorporated into a growing strand of viral DNA, causing chain termination due to the absence of the 3′-hydroxyl group. They can also inhibit the viral enzyme reverse transcriptase.

It has been recognized that drug-resistant variants of HIV can emerge after prolonged treatment with an antiviral agent. Drug resistance most typically occurs by mutation of a gene that encodes for an enzyme used in viral replication, and most typically in the case of HIV, reverse transcriptase, protease, or DNA polymerase. Recently, it has been demonstrated that the efficacy of a drug against HIV infection can be prolonged, augmented, or restored by administering the compound in combination or alternation with a second, and perhaps third, antiviral compound that induces a different mutation from that caused by the principle drug. Alternatively, the pharmacokinetics, biodistribution, or other parameter of the drug can be altered by such combination or alternation therapy. In general, combination therapy is typically preferred over alternation therapy because it induces multiple simultaneous pressures on the virus.

A number of HIV patients exposed to various drug treatment regimens have developed drug resistance, in the form of the K65R mutation in the HIV reverse transcriptase. The rate of the K65R mutation has steadily increased over time among treatment-experienced patients, and currently is above 4%. This fact is directly related to the wide use of tenofovir in clinical practice. Patients on tenofovir-containing triple nucleoside regimens have experienced high rates of early virological failure associated with this mutation. In addition to tenofovir, K65R is selected in vitro by zalcitabine (Hivid), didanosine (Videx), stavudine (Zerit), and abacavir (Ziagen). K65R reduces the susceptibility to these nucleoside analogues, but retains the activity of zidovudine (Retrovir) and other thymidine nucleosides.

Although patients with the K65R mutation can be treated with zidovudine (AZT), the approved AZT oral dose, 300 mg bid, is associated with bone marrow toxicity thought to be secondary to zidovudine-monophosphate (AZT-MP) accumulation.

Treatment for AIDS using attachment and fusion inhibitors as well as other antiviral drugs has been somewhat effective. Current clinical treatments for HIV-1 infections include triple drug combinations called Highly Active Antiretroviral Therapy (“HAART”). HAART typically involves various combinations of nucleoside reverse transcriptase inhibitors, non-nucleoside reverse transcriptase inhibitors, and HIV-1 protease inhibitors. In compliant patients, HAART is effective in reducing mortality and progression of HIV-1 infection to AIDS. However, these multidrug therapies do not eliminate HIV-1 and long-term treatment often results in multidrug resistance. Also, many of these drugs are highly toxic and/or require complicated dosing schedules that reduce compliance and limit efficacy. There is, therefore, a continuing need for the development of additional drugs for the prevention and treatment of HIV-1 infection and AIDS. Ideally, these drugs would target early stages in the HIV-1 replication cycle, i.e., inhibit or prevent attachment and fusion.

It would be useful to have combination therapy that minimizes the virological failure of patients taking nucleoside reverse transcriptase inhibitors that select for K65R. It would further be useful to have combination therapy for HIV or other retroviral infections which uses a lower, but effective dosage of zidovudine or other thymidine nucleoside reverse transcriptase inhibitors to minimize the side effects associated with normal dosage regimens for these agents. It would also be useful to provide a combination therapy that provides a cure for HIV/AIDS, by destroying the virus altogether in all its reservoirs. The present invention provides such combination therapy, as well as methods of treatment using the therapy.

### SUMMARY OF THE INVENTION

Combinations of antiretroviral nucleoside reverse transcriptase inhibitors, and methods for their use in treating retroviral infections, are provided.

In one embodiment, the combinations include a) zidovudine (AZT) or other thymidine nucleoside antiretroviral agents, and b) non-thymidine nucleoside antiretroviral agents, such as tenofovir, abacavir, (−)-β-D-2-aminopurine dioxolane (APD) and DAPD, which can select for the K65R mutation. In this embodiment, the dosage of AZT or other thymidine nucleoside antiretroviral agents is lower than conventional dosages, in order to reduce side effects, while still maintaining an efficacious therapeutic level of the therapeutic agent. For example, to minimize side effects associated with administration of AZT, such as bone marrow toxicity resulting in anemia, one can effectively lower the dosage to somewhere between around 100 and around 250 mg bid, preferably around 200 mg bid.

Using the lower (but still effective) dosage of AZT, one can minimize bone marrow toxicity believed to be secondary to zidovudine-monophosphate (AZT-MP) accumulation by significantly lowering the amount of AZT-MP present in the patient, without significant changes in the levels of zidovudine-triphosphate (AZT-TP), responsible for antiviral activity.

In another embodiment, the combinations include zidovudine (AZT) or other thymidine nucleoside antiretroviral agents, and DAPD or APD. In this embodiment, the dosage of AZT or other thymidine nucleoside antiretroviral agents can be the same as or lower than conventional dosages.

In a third embodiment, the combinations include at least one adenine nucleoside antiviral agent, at least one cytosine nucleoside antiviral agent, at least one guanine nucleoside antiviral agent, and at least one thymidine nucleoside antiviral agent. In one aspect of this embodiment, the therapeutic combinations include, and further include at least one additional agent selected from reverse transcriptase inhibitors, especially non-nucleoside viral polymerase inhibitors, protease inhibitors, fusion inhibitors, entry inhibitors, attachment inhibitors, and integrase inhibitors such as raltegravir (Isentress) or MK-0518, GS-9137 (elvitegravir, Gilead Sciences), GS-8374 (Gilead Sciences), or GSK-364735.

It is believed that this therapy, particularly when administered at an early stage in the development of HIV infection, has the possibility of eliminating HIV infection in a patient. That is, the presence of the different nucleosides containing all the possible bases (ACTG) and additional agents minimizes the ability of the virus to adapt its reverse transcriptase and develop resistance to any class of nucleoside antiviral nucleosides (i.e., adenine, cytosine, thymidine, or guanine), because it would be susceptible to at least one of the other nucleoside antiviral agents that are present, and/or the additional non-NRTI therapeutic agent. Furthermore, hitting the same target such as the active site of the HIV polymerase with different bases allows complete and thorough chain termination of all the possible growing viral DNA chains. The use of an NNRTI in addition to the four different nucleosides (ACTG analogs) could be even more effective since NNRTI bind to the HIV-polymerase and cause the enzyme to change conformation preventing chain elongation by natural nucleosides interacting in the active site of the enzyme.

In any of these embodiments, additional therapeutic agents can be used in combination with these agents, particularly including agents with a different mode of attack. Such agents include but are not limited to: antivirals, such as cytokines, e.g., rIFN alpha, rIFN beta, rIFN gamma; amphotericin B as a lipid-binding molecule with anti-HIV activity; a specific viral mutagenic agent (e.g., ribavirin), an HIV VIF inhibitor, and an inhibitor of glycoprotein processing.

In any of these embodiments, the various individual therapeutic agents, such as the zidovudine (ZDV, AZT) or other thymidine nucleoside antiretroviral agent and non-thymidine nucleoside antiretroviral agents which select for the K65R mutation in the first embodiment, can be administered in combination or in alternation. When administered in combination, the agents can be administered in a single or in multiple dosage forms. In some embodiments, some of the antiviral agents are orally administered, whereas other antiviral agents are administered by injection, which can occur at around the same time, or at different times.

The invention encompasses combinations of the two types of antiviral agents, or pharmaceutically acceptable derivatives thereof, that are synergistic, i.e., better than either agent or therapy alone.

The antiviral combinations described herein provide means of treatment which can not only reduce the effective dose of the individual drugs required for antiviral activity, thereby reducing toxicity, but can also improve their absolute antiviral effect, as a result of attacking the virus through multiple mechanisms. That is, the combinations are useful because their synergistic actions permit the use of less drug, increase the efficacy of the drugs when used together in the same amount as when used alone. Similarly, the novel antiviral combinations provide a means for circumventing the development of viral resistance to a single therapy, thereby providing the clinician with a more efficacious treatment.

The disclosed combination or alternation therapies are useful in the prevention and treatment of HIV infections and other related conditions such as AIDS-related complex (ARC), persistent generalized lymphadenopathy (PGL), AIDS-related neurological conditions, anti-HIV antibody positive and HIV-positive conditions, Kaposi's sarcoma, thrombocytopenia purpurea and opportunistic infections. In addition, these compounds or formulations can be used prophylactically to prevent or retard the progression of clinical illness in individuals who are anti-HIV antibody or HIV-antigen positive or who have been exposed to HIV. For example, the compositions can prevent or retard the development of K65R resistant HIV. The therapy can be also used to treat other viral infections, such as HIV-2.

### BRIEF DESCRIPTION OF THE FIGURES

FIG. 1 is a graph showing predicted plasma concentrations of AZT (mean±SD) for 7 days in simulated individuals (n=3,000), given AZT 200 mg bid (grey) or 300 mg bid (black).

FIG. 2 is a graph showing predicted cellular levels of AZT-MP per 10⁶ cells (mean±SD) for 7 days for simulated individuals (n=3,000) given AZT 200 mg bid (grey) or 300 mg bid (black).

FIG. 3 is a graph showing predicted levels of AZT-TP per 10⁶ cells (mean±SD) for 7 days in simulated individuals (n=3,000) given AZT 200 mg bid (grey) or 300 mg bid (black). (Mean−SD not shown since <0.)

FIG. 4A is a representative histogram from three separate simulations of maximal AZT-MP levels for individuals given 200 mg bid (grey) or 300 mg (dark grey) (n=3,000 per simulated trial).

FIG. 4B is a representative histogram from three separate simulations of maximal AZT-TP levels for individuals given 200 mg bid (grey) or 300 mg (dark grey) (n=3,000 per simulated trial).

FIG. 5 is a graph showing the predicted maximal cellular concentrations of AZT-TP (mean ()±SD and median (X)) versus dose (mg bid) in simulated individuals (n=3,000 per simulated trial).

FIG. 6 is a graph showing the mean change in hemoglobin (g/dL) from baseline, in terms of treatment and days, for treatment with 500 mg bid DAPD, 500 mg bid DAPD and 200 mg bid AZT, and 500 mg bid DAPD and 300 mg bid AZT.

FIG. 7 is a graph showing the mean change in MCV (femtoliters, +/−SD), in terms of treatment and days, for treatment with 500 mg bid DAPD, 500 mg bid DAPD and 200 mg bid AZT, and 500 mg bid DAPD and 300 mg bid AZT.

### DETAILED DESCRIPTION OF THE INVENTION

The present invention is directed to compositions and methods for treating viral infections, such as HIV infections. The various embodiments of the invention are described in more detail below, and will be better understood with reference to the following non-limiting definitions.

### DEFINITIONS

As used herein, the term antiviral nucleoside agent refers to antiviral nucleosides that have anti-HIV activity. The agents can be active against other viral infections as well, so long as they are active against HIV.

The term “antiviral thymidine nucleosides” refers to thymidine analogues with anti-HIV activity, including but not limited to, AZT (zidovudine) and D4T (2′,3′-didehydro-3′ deoxythymidine (stravudine), and 1-β-D-Dioxolane)thymine (DOT) or their prodrugs.

The term “antiviral guanine nucleosides” refers to guanine analogues with anti-HIV activity, including but not limited to, HBG [9-(4-hydroxybutyl)guanine], lobucavir ([1R(1alpha,2beta,3alpha)]-[2,3-bis(hydroxymethyl)cyclobutyl]guanine), abacavir ((1S,4R)-4-[2-Amino-6-(cyclopropylamino)-9H-purin-9-yl]-2-cyclopentene-1-methanol sulfate (salt), a prodrug of a G-carbocyclic nucleoside) and additional antiviral guanine nucleosides disclosed in U.S. Pat. No. 5,994,321

The term “antiviral cytosine nucleosides” refers to cytosine analogues with anti-HIV activity, including but not limited to, (−)-2′,3′-dideoxy-3′-thiacytidine (3TC) and its 5-fluoro analog (FTC, Emtricitaine), 2′,3′-dideoxycytidine (DDC), Racivir, β-D-2′,3′-didehydro-2′,3′-dideoxy-5-fluorocytidine (DFC, D-d4FC, RVT, Dexelvucitabine) and its enantiomer L-D4FC, and apricitabine (APC, AVX754, BCH-10618).

The term “antiviral adenine nucleosides” refers to adenine analogues with anti-HIV activity, including, but not limited to 2′,3′-dideoxy-adenosine (ddAdo), 2′,3′-dideoxyinosine (DDI), 9-(2-phosphonylmethoxyethyl)adenine (PMEA), 9-R-2-phosphonomethoxypropyl adenine (PMPA, Tenofovir) (K65R is resistant to PMPA), Tenofovir disoproxil fumarate (9-[(R)-2[[bis[[isopropoxycarbonyl)oxy]-methoxy]-phosphinyl]methoxy]propyl]adenine fumarate, TDF), bis(isopropyloxymethylcarbonyl)PMPA [bis(poc)PMPA], GS-9148 (Gilead Sciences) as well as those disclosed in Balzarini, J.; De Clercq, E. Acyclic purine nucleoside phosphonates as retrovirus inhibitors. In: Jeffries D J, De Clercq E., editors. Antiviral chemotherapy. New York, N.Y.: John Wiley & Sons, Inc.; 1995. pp. 41-45, the contents of which are hereby incorporated by reference.

The term AZT is used interchangeably with the term zidovudine throughout. Similarly, abbreviated and common names for other antiviral agents are used interchangeably throughout.

As used herein, the term DAPD ((2R,4R)-2-amino-9-[(2-hydroxymethyl)-I,3-dioxolan-4-yl]adenine) is also intended to include a related form of DAPD known as APD [(−)-β-D-2-aminopurine dioxolane]. All optically active forms of DAPD are intended to be within the scope of the invention described herein, including optically active forms and racemic forms.

As used herein, the term “pharmaceutically acceptable salts” refers to pharmaceutically acceptable salts which, upon administration to the recipient, are capable of providing directly or indirectly, a nucleoside antiviral agent, or that exhibit activity themselves.

As used herein, the term “prodrug” refers to the 5′ and N-acylated, alkylated, or phosphorylated (including mono, di, and triphosphate esters as well as stabilized phosphates and phospholipid) derivatives of AZT or a non-thymidine nucleoside antiviral agent. In one embodiment, the acyl group is a carboxylic acid ester in which the non-carbonyl moiety of the ester group is selected from straight, branched, or cyclic alkyl, alkoxyalkyl including methoxymethyl, aralkyl including benzyl, aryloxyalkyl including phenoxymethyl, aryl including phenyl optionally substituted by halogen, alkyl, alkyl or alkoxy, sulfonate esters such as alkyl or aralkyl sulphonyl including methanesulfonyl, trityl or monomethoxytrityl, substituted benzyl, trialkylsilyl, or diphenylmethylsilyl. Aryl groups in the esters optimally comprise a phenyl group. The alkyl group can be straight, branched or cyclic and is preferably C₁₋₁₈.

As used herein, the term “resistant virus” refers to a virus that exhibits a three, and more typically, five or greater fold increase in EC₅₀ compared to naive virus in a constant cell line, including, but not limited to peripheral blood mononuclear (PBM) cells, or MT2 or MT4 cells.

As used herein, the term “substantially pure” or “substantially in the form of one optical isomer” refers to a nucleoside composition that includes at least 95% to 98%, or more, preferably 99% to 100%, of a single enantiomer of that nucleoside. In a preferred embodiment, AZT is administered in substantially pure form for any of the disclosed indications.

### I. Combinations of Thymidine Nucleoside Antiviral Agents and Non-Thymidine Nucleoside Antiviral Agents

In one embodiment, the compositions include both thymidine nucleoside antiviral agents and non-thymidine nucleoside antiviral agents, where the non-thymidine nucleoside antiviral agents select for the K65R mutation. Representative agents that select for the K65 mutation include tenofovir, and DAPD.

The thymidine nucleoside antiviral agent is administered in combination or alternation with the non-thymidine nucleoside antiviral agent in a manner in which both agents act synergistically against the virus. The compositions and methods described herein can be used to treat patients infected with a drug resistant form of HIV, specifically, a form including the K65R mutation.

In this embodiment, the dosage of the thymidine nucleoside antiviral agent, such as AZT, is lower than that commonly associated with side effects, but high enough to elicit favorable antiviral activity. Mechanistic studies suggest that the sub-linear increases in AZT-TP observed at higher doses of AZT may be explained by saturation of thymidylate kinase enzyme. Thus, it is believed that when too much of the agent is administered, the capacity of phosphorylating enzymes that produce the active triphosphate form of the agents becomes saturated, so that a maximal amount of the triphosphate is formed until the enzyme is again ready to convert the agent to the triphosphate form. Excess agent is converted to a monophosphate, which is then accumulated, and it is the monophosphate that is believed to result in side effects such as bone marrow toxicity. Therefore, it can be important to strike a balance between the amount of drug that can be effectively delivered and the amount of drug that results in side effects.

A previous clinical study (data not shown) suggested that AZT 100 mg tid produces significantly lower plasma AZT and lymphocyte AZT-MP levels, without significant changes in the levels of zidovudine-triphosphate (AZT-TP), responsible for antiviral activity.

A simulation study was performed in silico to optimize the AZT dose for bid administration with K65R-selecting antiretroviral agents in virtual subjects using population pharmacokinetic and cellular enzyme kinetic parameters of AZT. These simulations predicted that AZT 200 mg bid produces similar AZT-TP levels associated with antiviral efficacy, >91% overlap of maximal cellular levels with AZT 300 mg bid, and reduced AZT-MP levels associated with toxicity, <23% overlap with AZT 300 mg bid. These in silico findings suggest that AZT 200 mg bid can maintain antiviral efficacy, while producing lower toxicity and delivering anti-K65R activity. The study is described in more detail in Example 1.

The in silico and in vivo data demonstrate that lower dosage AZT (i.e., between around 100 and around 250 bid) can be effective, yet minimize the accumulation of toxic by-products such as the monophosphate form of the agents.

Further, the combination of the thymidine antiviral nucleoside agents, such as AZT, help prevent the development of viral resistance to other antiviral agents. That is, data from large genotype databases suggest that various non-thymidine nucleoside reverse transcriptase inhibitors, such as tenofovir, DXG and DAPD, can select for the K65R resistance mutation in HIV-1 infected individuals. Studies performed in vitro and in vivo suggest that viruses containing the K65R mutation remain susceptible to zidovudine (AZT) and other thymidine nucleoside antiretroviral agents. Therefore, co-formulation of AZT with these agents as a “resistance repellent” for the K65R mutation provides better therapy than either alone.

### I. Combinations of Thymidine Nucleoside Antiviral Agents and DAPD

In another embodiment, the combinations include zidovudine (AZT) or other thymidine nucleoside antiretroviral agents, and DAPD. In this embodiment, the dosage of AZT or other thymidine nucleoside antiretroviral agents can be the same as or lower than conventional dosages.

As discussed above with respect to the first embodiment, co-formulation of AZT with other antiviral nucleoside agents as a “resistance repellent” for the K65R mutation provides better therapy than either alone. AZT and other thymidine nucleoside antiviral agents are also associated with various mutations in the viral DNA, and, therefore resistance to AZT can develop. These mutations are known as thymidine analog mutations (TAMs).

Amdoxovir (AMDX; DAPD) has been well studied in six trials in close to 200 subjects. AZT is synergistic with DAPD and prevents selection of K65R and thymidine analog mutations (TAMs). That is, while the AZT reduces the ability of the virus to develop the K65R mutation following administration of DAPD, the DAPD reduces the ability of the virus to develop TAMs mutations following administration of AZT. Thus, the two agents administered together are superior to either administered alone, since they can each effectively reduce the presence of viral mutations that would render the other either ineffective or less effective as an anti-HIV agent.

Further, as is the case in the first embodiment discussed above, the dosage of AZT can be reduced in a manner which reduces the amount of AZT monophosphate (AZT-MP) accumulation, while maintaining antiviral effect. Thus, while AZT can be administered in the conventional dosage of 300 mg bid, it can also be administered in a lower dosage (i.e., between around 100 and around 250 bid) can be effective, yet minimize the accumulation of toxic by-products such as the monophosphate form of the agents.

The results of a clinical study are shown in Example 2, where the dosage of DAPD was 500 mg bid, and the dosage of AZT in some patients was 300 mg bid, and in other patients was 200 mg bid, for 10 days. In each arm, subjects were randomized 3:1 to DAPD: placebo. Viral loads were determined daily. DAPD/AZT viral load decline indicated synergy, and the combination therapy was effective and well tolerated. It is believed that long term studies with lower dose AZT will demonstrate decreased toxicity as well, though this study was limited to 10 days.

In this study, the effect of the combination therapy on hemoglobin concentrations and mean corpuscular volume, an indicator of the susceptibility to bone marrow toxicity, was determined. Twenty-four subjects were enrolled in a study (shown in Example 3) using the dosages for DAPD and AZT discussed above. Hematological indices including hemoglobin (g/dl) and mean corpuscular volume (MCV, femtoliters) were measured over time, and the data showed that the trend in decrease in hemoglobin from Baseline was DAPD/AZT 300≧AZT 300≧DAPD/AZT 200>AZT 200>DAPD>placebo and the trend in increase in MCV from Baseline was DAPD/AZT 300>AZT 300>DAPD/AZT 200>AZT 200>placebo>DAPD. These data in humans shows that the lower dosage of AZT effectively lowers the incidence of side effects associated with bone marrow toxicity.

III. Combination Therapy with a Combination of Adenine, Cytosine, Thymidine, and Guanine Nucleoside Antiviral Agents

In a third embodiment, a combination therapy is administered which has the capability of attacking HIV in a variety of mechanisms. That is, the combination therapy includes an effective amount of at least one adenine, cytosine, thymine, and guanosine nucleoside antiviral, as well as one or more additional agents other than NRTI that inhibit HIV viral loads via a different mechanism. Examples include reverse transcriptase inhibitors, protease inhibitors, fusion inhibitors, entry inhibitors, attachment inhibitors, polymerase inhibitors, and integrase inhibitors such as integrase inhibitors such as raltegravir (Isentress) or MK-0518, GS-9137 (Gilead Sciences), GS-8374 (Gilead Sciences), or GSK-364735.

It is believed that this therapy, particularly when administered at an early stage in the development of HIV infection, has the possibility of eliminating HIV infection in a patient. That is, the presence of the different nucleosides and additional agents minimizes the ability of the virus to adapt its reverse transcriptase and develop resistance to any class of nucleoside antiviral nucleosides (i.e., adenine, cytosine, thymidine, or guanine), because it would be susceptible to at least one of the other nucleoside antiviral agents that are present, and/or the additional non-NRTI therapeutic agent. In addition the lipophilic character of certain agents would allow them to penetrate certain compartments where virus could replicate (e.g., brain, testicles, gut).

Representative agents are described in more detail below.

Attachment and Fusion Inhibitors

Attachment and fusion inhibitors are anti-HIV drugs which are intended to protect cells from infection by HIV by preventing the virus from attaching to a new cell and breaking through the cell membrane. These drugs can prevent infection of a cell by either free virus (in the blood) or by contact with an infected cell. These agents are susceptible to digestive acids, so are commonly delivered by break them down, most of these drugs are given by injections or intravenous infusion.

Examples are shown in the table that follows:

Entry Inhibitors (including Fusion Inhibitors)

| Brand | Generic | | Experimental | Pharmaceutical |
|---|---|---|---|---|
| Name | Name | Abbreviation | Code | Company |
| Fuzeon ™ | enfuvirtide | | T-20 | Trimeris |
| | | | T-1249 | Trimeris |
| | | | AMD-3100 | AnorMED, Inc. |
| | CD4-IgG2 | | PRO-542 | Progenics |
| | | | | Pharmaceuticals |
| | | | BMS-488043 | Bristol-Myers |
| | | | | Squibb |
| | aplaviroc | | GSK-873, 140 | GlaxoSmithKline |
| | Peptide T | | | Advanced |
| | | | | Immuni T, Inc. |
| | | | TNX-355 | Tanox, Inc. |
| | maraviroc | | UK-427, 857 | Pfizer |

CXCR4 Inhibitor

| | | |
|---|---|---|
| AMD070 | AMD11070 | AnorMED, Inc. |

CCR5 antagonist

| | | | |
|---|---|---|---|
| vicriroc | SCH-D | SCH-417690 | Schering-Plough |

Additional fusion and attachment inhibitors in human trials include AK602, AMD070, BMS-378806, HGS004, INCB9471, PRO140, Schering C, SP01A, and TAK-652.

AK602 is a CCR5 blocker being developed by Kumamoto University in Japan.

AMD070 by AnorMed blocks the CXCR4 receptor on CD4 T-cells to inhibit HIV fusion.

BMS-378806 is an attachment inhibitor that attaches to gp120, a part of the HIV virus.

HGS004 by Human Genome Sciences, is a monoclonal antibody CCR5 blocker.

INCB 9471 is sold by Incyte Corporation.

PRO 140 by Progenics blocks fusion by binding to a receptor protein on the surface of CD4 cells.

SP01A by Samaritan Pharmaceuticals is an HIV entry inhibitor.

TAK-652 by Takeda blocks binding to the CCR5 receptor.

Polymerase Inhibitors

The DNA polymerization activity of HIV-1 reverse transcriptase (RT) can be inhibited by at least three mechanistically distinct classes of compounds. Two of these are chain terminating nucleoside analogs (NRTIs) and allosteric non-nucleoside RT inhibitors (NNRTIs). The third class includes pyrophosphate mimetics such as foscarnet (phosphonoformic acid, PFA).

The reverse transcriptase has a second enzymatic activity, ribonuclease H (RNase H) activity, which maps to a second active site in the enzyme. RNase H activity can be inhibited by various small molecules (polymerase inhibitors). Examples include diketo acids, which bind directly to the RNase H domain, or compounds like PFA, which are believed to bind in the polymerase domain.

Examples of these compounds are listed in the tables that follow.

HIV Therapies: Nucleoside/Nucleotide Reverse
Transcriptase Inhibitors (NRTIs)

| | | | Experimental | Pharmaceutical |
|---|---|---|---|---|
| Brand Name | Generic Name | Abbreviation | Code | Company |
| Retrovir ® | zidovudine | AZT or ZDV | | GlaxoSmithKline |
| Epivir ® | lamivudine | 3TC | | GlaxoSmithKline |
| Combivir ® | zidovudine + | AZT + 3TC | | GlaxoSmithKline |
| | lamivudine | | | |
| Trizivir ® | abacavir + | ABC + AZT + 3TC | | GlaxoSmithKline |
| | zidovudine + | | | |
| | lamivudine | | | |
| Ziagen ® | abacavir | ABC | 1592U89 | GlaxoSmithKline |
| Epzicom ™ | abacavir + | ABC + 3TC | | GlaxoSmithKline |
| | lamivudine | | | |
| Hivid ® | zalcitabine | ddC | | Hoffmann-La |
| | | | | Roche |
| Videx ® | didanosine: | ddI | BMY-40900 | Bristol-Myers |
| | buffered | | | Squibb |
| | versions | | | |
| Entecavir | baraclude | | | Bristol-Myers |
| | | | | Squibb |
| Videx ® EC | didanosine: | ddI | | Bristol-Myers |
| | delayed- | | | Squibb |
| | release | | | |
| | capsules | | | |
| Zerit ® | stavudine | d4T | BMY-27857 | Bristol-Myers |
| | | | | Squibb |
| Viread ™ | tenofovir | TDF or | | Gilead Sciences |
| | disoproxil | Bis(POC) | | |
| | fumarate (DF) | PMPA | | |
| Emtriva ® | emtricitabine | FTC | | Gilead Sciences |
| Truvada ® | Viread + | TDF + FTC | | Gilead Sciences |
| | Emtriva | | | |
| Atripla ™ | | TDF + FTC + | | Gilead/BMS/Merck |
| | | Sustiva ® | | |
| | Amdoxovir | DAPD, AMDX | | RFS Pharma LLC |
| apricitabine | AVX754 | | SPD 754 | Avexa Ltd |
| | Alovudine | FLT | MIW-310 | Medivir |
| | Elvucitabine | L-FD4C | ACH-126443, | Achillion |
| | KP-1461 | | SN1461, | Koronis |
| | | | SN1212 | |
| | Racivir | RCV | | Pharmasset |
| | | DOT | | Pharmasset |
| Dexelvucitabine | Reverset | D-D4FC, DFC | DPC 817 | Pharmasset/Emory |
| | | | | University |
| | | | GS9148 and | Gilead Sciences |
| | | | prodrugs | |
| | | | thereof | |

HIV Therapies: Non-Nucleoside Reverse
Transcriptase Inhibitors (NNRTIs)

| Brand | Generic | | Experimental | Pharmaceutical |
|---|---|---|---|---|
| Name | Name | Abbreviation | Code | Company |
| Viramune ® | nevirapine | NVP | BI-RG-587 | Boehringer Ingelheim |
| Rescriptor ® | delavirdine | DLV | U-90152S/T | Pfizer |
| Sustiva ® | efavirenz | EFV | DMP-266 | Bristol-Myers Squibb |
| | (+)-calanolide | | | Sarawak Medichem |
| | A | | | |
| | capravirine | CPV | AG-1549 or S-1153 | Pfizer |
| | | | DPC-083 | Bristol-Myers Squibb |
| | | | TMC-125 | Tibotec-Virco Group |
| | | | TMC-278 | Tibotec-Virco Group |
| | | | IDX12899 | Idenix |
| | | | IDX12989 | Idenix |
| RDEA806 | | | | Ardea Bioscience, Inc. |

Protease Inhibitors

Protease inhibitors treat or prevent HIV infection by preventing viral replication. They act by inhibiting the activity of HIV protease, an enzyme that cleaves nascent proteins for final assembly of new virons. Examples are shown in the table that follows.

HIV Therapies: Protease Inhibitors (PIs)

| Brand | Generic | | Experimental | Pharmaceutical |
|---|---|---|---|---|
| Name | Name | Abbreviation | Code | Company |
| Invirase ® | saquinavir (Hard | SQV (HGC) | Ro-31-8959 | Hoffmann-La Roche |
| | Gel Cap) | | | |
| Fortovase ® | saquinavir (Soft | SQV (SGC) | | Hoffmann-La Roche |
| | Gel Cap) | | | |
| Norvir ® | ritonavir | RTV | ABT-538 | Abbott Laboratories |
| Crixivan ® | indinavir | IDV | MK-639 | Merck & Co. |
| Viracept ® | nelfinavir | NFV | AG-1343 | Pfizer |
| Agenerase ® | amprenavir | APV | 141W94 or | GlaxoSmithKline |
| | | | VX-478 | |
| Kaletra ® | lopinavir + | LPV | ABT-378/r | Abbott Laboratories |
| | ritonavir | | | |
| Lexiva ® | fosamprenavir | | GW-433908 or | GlaxoSmithKline |
| | | | VX-175 | |
| Aptivus ® | tripanavir | TPV | PNU-140690 | Boehringer Ingelheim |
| Reyataz ® | atazanavir | | BMS-232632 | Bristol-Myers Squibb |
| | brecanavir | | GW640385 | GlaxoSmithKline |
| Prezista ™ | darunavir | | TMC114 | Tibotec |

HIV Therapies: Other Classes of Drugs

| Brand | Generic | | Experimental | Pharmaceutical |
|---|---|---|---|---|
| Name | Name | Abbreviation | Code | Company |
| Viread ™ | tenofovir | TDF or | | Gilead Sciences |
| | disoproxil | Bis(POC) | | |
| | fumarate | PMPA | | |
| | (DF) | | | |

Cellular Inhibitors

| Brand | Generic | | Experimental | Pharmaceutical |
|---|---|---|---|---|
| Name | Name | Abbreviation | Code | Company |
| Droxia ® | hydroxyurea | HU | | Bristol-Myers |
| | | | | Squibb |

HIV Therapies: Immune-Based Therapies

| | | | Experimental | |
|---|---|---|---|---|
| Brand Name | Generic Name | Abbreviation | Code | Pharmaceutical Company |
| Proleukin ® | aldesleukin, or | IL-2 | | Chiron |
| | Interleukin-2 | | | Corporation |
| Remune ® | HIV-1 Immunogen, | | AG1661 | The Immune |
| | or Salk vaccine | | | Response Corporation |
| | | | HE2000 | HollisEden Pharmaceuticals |

### IV. Combination or Alternation HIV-Agents

In general, during alternation therapy, an effective dosage of each agent is administered serially, whereas in combination therapy, an effective dosage of two or more agents are administered together. In alternation therapy, for example, one or more first agents can be administered in an effective amount for an effective time period to treat the viral infection, and then one or more second agents substituted for those first agents in the therapy routine and likewise given in an effective amount for an effective time period.

The dosages will depend on such factors as absorption, biodistribution, metabolism and excretion rates for each drug as well as other factors known to those of skill in the art. It is to be noted that dosage values will also vary with the severity of the condition to be alleviated. It is to be further understood that for any particular subject, specific dosage regimens and schedules should be adjusted over time according to the individual need and the professional judgment of the person administering or supervising the administration of the compositions.

Examples of suitable dosage ranges for anti-HIV compounds, including thymidine nucleoside derivatives such as AZT and non-thymidine nucleoside derivatives such as 3TC, can be found in the scientific literature and in the Physicians Desk Reference. Many examples of suitable dosage ranges for other compounds described herein are also found in public literature or can be identified using known procedures. These dosage ranges can be modified as desired to achieve a desired result.

In one preferred embodiment, AZT is administered in combination with a non-thymidine nucleoside antiviral agent that selects for the K65R mutation. In particular embodiments, AZT is administered in combination or alternation with tenofovir, APD, or DAPD.

### V. Pharmaceutical Compositions

Humans suffering from effects caused by any of the diseases described herein, and in particular, HIV infection, can be treated by administering to the patient an effective amount of the compositions described above, in the presence of a pharmaceutically acceptable carrier or diluent, for any of the indications or modes of administration as described in detail herein. The active materials can be administered by any appropriate route, for example, orally, parenterally, enterally, intravenously, intradermally, subcutaneously, transdermally, intranasally or topically, in liquid or solid form.

The active compounds are included in the pharmaceutically acceptable carrier or diluent in an amount sufficient to deliver to a patient a therapeutically effective amount of compound to inhibit viral replication in vivo, especially HIV replication, without causing serious toxic effects in the treated patient. By “inhibitory amount” is meant an amount of active ingredient sufficient to exert an inhibitory effect as measured by, for example, an assay such as the ones described herein.

A preferred dose of the compound for all the above-mentioned conditions will be in the range from about 1 to 75 mg/kg, preferably 1 to 20 mg/kg, of body weight per day, more generally 0.1 to about 100 mg per kilogram body weight of the recipient per day. The effective dosage range of the pharmaceutically acceptable derivatives can be calculated based on the weight of the parent nucleoside or other agent to be delivered. If the derivative exhibits activity in itself, the effective dosage can be estimated as above using the weight of the derivative, or by other means known to those skilled in the art.

The compounds are conveniently administered in unit any suitable dosage form, including but not limited to one containing 7 to 3000 mg, preferably 70 to 1400 mg of active ingredient per unit dosage form. An oral dosage of 50 to 1000 mg is usually convenient.

Ideally, the active ingredient should be administered to achieve peak plasma concentrations of the active compound of from about 0.02 to 70 micromolar, preferably about 0.5 to 10 micromolar. This may be achieved, for example, by the intravenous injection of a 0.1 to 25% solution of the active ingredient, optionally in saline, or administered as a bolus of the active ingredient.

The concentration of active compound in the drug composition will depend on absorption, distribution, metabolism and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient may be administered at once, or may be divided into a number of smaller doses to be administered at varying intervals of time.

A preferred mode of administration of the active compound is oral. Oral compositions will generally include an inert diluent or an edible carrier. They may be enclosed in gelatin capsules or compressed into tablets. For the purpose of oral therapeutic administration, the active compound can be incorporated with excipients and used in the form of tablets, troches, or capsules. Pharmaceutically compatible bind agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or other enteric agents.

The compounds can be administered as a component of an elixir, suspension, syrup, wafer, chewing gum or the like. A syrup may contain, in addition to the active compounds, sucrose as a sweetening agent and certain preservatives, dyes and colorings and flavors.

The compounds or their pharmaceutically acceptable derivative or salts thereof can also be mixed with other active materials that do not impair the desired action, or with materials that supplement the desired action, such as antibiotics, antifungals, antiinflammatories, protease inhibitors, or other nucleoside or non-nucleoside antiviral agents, as discussed in more detail above. Solutions or suspensions used for parental, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation can be enclosed in ampoules, disposable syringes or multiple dose vials made of glass or plastic.

If administered intravenously, preferred carriers are physiological saline or phosphate buffered saline (PBS).

Liposomal suspensions (including liposomes targeted to infected cells with monoclonal antibodies to viral antigens) are also preferred as pharmaceutically acceptable carriers, these may be prepared according to methods known to those skilled in the art, for example, as described in U.S. Pat. No. 4,522,811. For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound or its monophosphate, diphosphate, and/or triphosphate derivatives is then introduced into the container. The container is then swirled by hand to free lipid material from the sides of the container and to disperse lipid aggregates, thereby forming the liposomal suspension.

In one embodiment, the composition is a co-formulated pill, tablet, or other oral drug delivery vehicle including DAPD plus AZT, with AZT at 200 mg and DAPD at 500 mg.

In another embodiment, this co-formulation of DAPD and AZT can be co-administered with ATRIPLA® (efavirenz 600 mg/emtricitabine (FTC) 200 mg/tenofovir disoproxil fumarate 300 mg). Because efavirenz is an NNRTI, tenofovir is an adenine nRTI, FTC is a cytosine nRTI, and AZT is a thymidine nRTI, and DAPD is deaminated in vivo to form DXG (a guanine nRTI), the combination of the coformulated DAPD plus AZT pill will provide all four bases (ACTG) plus an additional agent capable of interacting with HIV in a different mechanism.

Controlled Release Formulations

All of the U.S. patents cited in this section on controlled release formulations are incorporated by reference in their entirety.

The field of biodegradable polymers has developed rapidly since the synthesis and biodegradability of polylactic acid was reported by Kulkarni et al., in 1966 (“Polylactic acid for surgical implants,” Arch. Surg., 93:839). Examples of other polymers which have been reported as useful as a matrix material for delivery devices include polyanhydrides, polyesters such as polyglycolides and polylactide-co-glycolides, polyamino acids such as polylysine, polymers and copolymers of polyethylene oxide, acrylic terminated polyethylene oxide, polyamides, polyurethanes, polyorthoesters, polyacrylonitriles, and polyphosphazenes. See, for example, U.S. Pat. Nos. 4,891,225 and 4,906,474 to Langer (polyanhydrides), U.S. Pat. No. 4,767,628 to Hutchinson (polylactide, polylactide-co-glycolide acid), and U.S. Pat. No. 4,530,840 to Tice, et al. (polylactide, polyglycolide, and copolymers). See also U.S. Pat. No. 5,626,863 to Hubbell, et al which describes photopolymerizable biodegradable hydrogels as tissue contacting materials and controlled release carriers (hydrogels of polymerized and crosslinked macromers comprising hydrophilic oligomers having biodegradable monomeric or oligomeric extensions, which are end capped monomers or oligomers capable of polymerization and crosslinking); and PCT WO 97/05185 filed by Focal, Inc. directed to multiblock biodegradable hydrogels for use as controlled release agents for drug delivery and tissue treatment agents.

Degradable materials of biological origin are well known, for example, crosslinked gelatin. Hyaluronic acid has been crosslinked and used as a degradable swelling polymer for biomedical applications (U.S. Pat. No. 4,957,744 to Della Valle et. al.; (1991) “Surface modification of polymeric biomaterials for reduced thrombogenicity,” Polym. Mater. Sci. Eng., 62:731 735]).

Many dispersion systems are currently in use as, or being explored for use as, carriers of substances, particularly biologically active compounds. Dispersion systems used for pharmaceutical and cosmetic formulations can be categorized as either suspensions or emulsions. Suspensions are defined as solid particles ranging in size from a few manometers up to hundreds of microns, dispersed in a liquid medium using suspending agents. Solid particles include microspheres, microcapsules, and nanospheres. Emulsions are defined as dispersions of one liquid in another, stabilized by an interfacial film of emulsifiers such as surfactants and lipids. Emulsion formulations include water in oil and oil in water emulsions, multiple emulsions, microemulsions, microdroplets, and liposomes. Microdroplets are unilamellar phospholipid vesicles that consist of a spherical lipid layer with an oil phase inside, as defined in U.S. Pat. Nos. 4,622,219 and 4,725,442 issued to Haynes. Liposomes are phospholipid vesicles prepared by mixing water-insoluble polar lipids with an aqueous solution. The unfavorable entropy caused by mixing the insoluble lipid in the water produces a highly ordered assembly of concentric closed membranes of phospholipid with entrapped aqueous solution.

U.S. Pat. No. 4,938,763 to Dunn, et al., discloses a method for forming an implant in situ by dissolving a nonreactive, water insoluble thermoplastic polymer in a biocompatible, water soluble solvent to form a liquid, placing the liquid within the body, and allowing the solvent to dissipate to produce a solid implant. The polymer solution can be placed in the body via syringe. The implant can assume the shape of its surrounding cavity. In an alternative embodiment, the implant is formed from reactive, liquid oligomeric polymers which contain no solvent and which cure in place to form solids, usually with the addition of a curing catalyst.

A number of patents disclose drug delivery systems that can be used to administer the combination of the thymidine and non-thymidine nucleoside antiviral agents, or prodrugs thereof. U.S. Pat. No. 5,749,847 discloses a method for the delivery of nucleotides into organisms by electrophoration. U.S. Pat. No. 5,718,921 discloses microspheres comprising polymer and drug dispersed there within. U.S. Pat. No. 5,629,009 discloses a delivery system for the controlled release of bioactive factors. U.S. Pat. No. 5,578,325 discloses nanoparticles and microparticles of non-linear hydrophilic hydrophobic multiblock copolymers. U.S. Pat. No. 5,545,409 discloses a delivery system for the controlled release of bioactive factors. U.S. Pat. No. 5,494,682 discloses ionically cross-linked polymeric microcapsules.

U.S. Pat. No. 5,728,402 to Andrx Pharmaceuticals, Inc. describes a controlled release formulation that includes an internal phase which comprises the active drug, its salt or prodrug, in admixture with a hydrogel forming agent, and an external phase which comprises a coating which resists dissolution in the stomach. U.S. Pat. Nos. 5,736,159 and 5,558,879 to Andrx Pharmaceuticals, Inc. discloses a controlled release formulation for drugs with little water solubility in which a passageway is formed in situ. U.S. Pat. No. 5,567,441 to Andrx Pharmaceuticals, Inc. discloses a once-a-day controlled release formulation. U.S. Pat. No. 5,508,040 discloses a multiparticulate pulsatile drug delivery system. U.S. Pat. No. 5,472,708 discloses a pulsatile particle based drug delivery system. U.S. Pat. No. 5,458,888 describes a controlled release tablet formulation which can be made using a blend having an internal drug containing phase and an external phase which comprises a polyethylene glycol polymer which has a weight average molecular weight of from 3,000 to 10,000. U.S. Pat. No. 5,419,917 discloses methods for the modification of the rate of release of a drug form a hydrogel which is based on the use of an effective amount of a pharmaceutically acceptable ionizable compound that is capable of providing a substantially zero-order release rate of drug from the hydrogel. U.S. Pat. No. 5,458,888 discloses a controlled release tablet formulation.

U.S. Pat. No. 5,641,745 to Elan Corporation, plc discloses a controlled release pharmaceutical formulation which comprises the active drug in a biodegradable polymer to form microspheres or nanospheres. The biodegradable polymer is suitably poly-D,L-lactide or a blend of poly-D,L-lactide and poly-D,L-lactide-co-glycolide. U.S. Pat. No. 5,616,345 to Elan Corporation plc describes a controlled absorption formulation for once a day administration that includes the active compound in association with an organic acid, and a multi-layer membrane surrounding the core and containing a major proportion of a pharmaceutically acceptable film-forming, water insoluble synthetic polymer and a minor proportion of a pharmaceutically acceptable film-forming water soluble synthetic polymer. U.S. Pat. No. 5,641,515 discloses a controlled release formulation based on biodegradable nanoparticles. U.S. Pat. No. 5,637,320 discloses a controlled absorption formulation for once a day administration. U.S. Pat. Nos. 5,580,580 and 5,540,938 are directed to formulations and their use in the treatment of neurological diseases. U.S. Pat. No. 5,533,995 is directed to a passive transdermal device with controlled drug delivery. U.S. Pat. No. 5,505,962 describes a controlled release pharmaceutical formulation.

Prodrug Formulations

AZT or any of the nucleosides or other compounds which are described herein for use in combination or alternation therapy with AZT or its related compounds can be administered as an acylated prodrug or a nucleotide prodrug, as described in detail below.

Any of the nucleosides described herein or other compounds that contain a hydroxyl or amine function can be administered as a nucleotide prodrug to increase the activity, bioavailability, stability or otherwise alter the properties of the nucleoside. A number of nucleotide prodrug ligands are known. In general, alkylation, acylation or other lipophilic modification of the hydroxyl group of the compound or of the mono, di or triphosphate of the nucleoside will increase the stability of the nucleotide. Examples of substituent groups that can replace one or more hydrogens on the phosphate moiety or hydroxyl are alkyl, aryl, steroids, carbohydrates, including sugars, 1,2-diacylglycerol and alcohols. Many are described in R. Jones and N. Bischofberger, Antiviral Research, 27 (1995) 1 17. Any of these can be used in combination with the disclosed nucleosides or other compounds to achieve a desire effect.

The active nucleoside or other hydroxyl containing compound can also be provided as an ether lipid (and particularly a 5′-ether lipid for a nucleoside), as disclosed in the following references, Kucera, L. S., N. Iyer, E. Leake, A. Raben, Modest E. K., D. L. W., and C. Piantadosi. 1990. “Novel membrane-interactive ether lipid analogs that inhibit infectious HIV-1 production and induce defective virus formation.” AIDS Res. Hum. Retroviruses. 6:491 501; Piantadosi, C., J. Marasco C. J., S. L. Morris-Natschke, K. L. Meyer, F. Gumus, J. R. Surles, K. S. Ishaq, L. S. Kucera, N. Iyer, C. A. Wallen, S. Piantadosi, and E. J. Modest. 1991. “Synthesis and evaluation of novel ether lipid nucleoside conjugates for anti-HIV activity.” J. Med. Chem. 34:1408.1414; Hosteller, K. Y., D. D. Richrnan D. A. Carson, L. M. Stuhmiller, G. M. T. van Wijk, and H. van den Bosch. 1992. “Greatly enhanced inhibition of human immunodeficiency virus type 1 replication in CEM and HT4-6C cells by 3′-deoxythymidine diphosphate dimyristoylglycerol, a lipid prodrug of 3′-deoxythymidine.” Antimicrob. Agents Chemother. 36:2025. 2029; Hostetler, K. Y., L. M. Stuhmiller, H. B. Lenting, H. van den Bosch, and D. D. Richman, 1990. “Synthesis and antiretroviral activity of phospholipid analogs of azidothymidine and other antiviral nucleosides.” J. Biol. Chem. 265:61127.

Nonlimiting examples of U.S. patents that disclose suitable lipophilic substituents that can be covalently incorporated into the nucleoside or other hydroxyl or amine containing compound, preferably at the 5′-OH position of the nucleoside or lipophilic preparations, include U.S. Pat. No. 5,149,794 (Sep. 22, 1992, Yatvin et al.); U.S. Pat. No. 5,194,654 (Mar. 16, 1993, Hostetler et al., U.S. Pat. No. 5,223,263 (Jun. 29, 1993, Hostetler et al.); U.S. Pat. No. 5,256,641 (Oct. 26, 1993, Yatvin et al.); U.S. Pat. No. 5,411,947 (May 2, 1995, Hostetler et al.); U.S. Pat. No. 5,463,092 (Oct. 31, 1995, Hostetler et al.); U.S. Pat. No. 5,543,389 (Aug. 6, 1996, Yatvin et al.); U.S. Pat. No. 5,543,390 (Aug. 6, 1996, Yatvin et al.); U.S. Pat. No. 5,543,391 (Aug. 6, 1996, Yatvin et al.); and U.S. Pat. No. 5,554,728 (Sep. 10, 1996; Basava et-al.), Foreign patent applications that disclose lipophilic substituents that can be attached to the nucleosides of the present invention, or lipophilic preparations, include WO 89/02733, WO 90/00555, WO 91/16920, WO 91/18914, WO 93/00910, WO 94/26273, WO 96/15132, EP 0 350 287, EP 93917054.4, and WO 91/19721.

Nonlimiting examples of nucleotide prodrugs are described in the following references: Ho, D. H. W. (1973) “Distribution of Kinase and deaminase of 1β-D-arabinofuranosylcytosine in tissues of man and muse.” Cancer Res. 33, 2816 2820; Holy, A. (1993) Isopolar phosphorous-modified nucleotide analogues,” In: De Clercq (Ed.), Advances in Antiviral Drug Design, Vol. I, JAI Press, pp. 179 231; Hong, C. I., Nechaev, A., and West, C. R. (1979a) “Synthesis and antitumor activity of 1β-D-arabino-furanosylcytosine conjugates of cortisol and cortisone.” Biochem. Biophys. Rs. Commun. 88, 1223 1229; Hong, C. I., Nechaev, A., Kirisits, A. J. Buchheit, D. J. and West, C. R. (1980) “Nucleoside conjugates as potential antitumor agents. 3. Synthesis and antitumor activity of 1-(β-D-arabinofuranosyl)cytosine conjugates of corticosteroids and selected lipophilic alcohols.” J. Med. Chem. 28, 171 177; Hosteller, K. Y., Stuhmiller, L. M., Lenting, H. B. M. van den Bosch, H. and Richman J Biol. Chem. 265, 6112 6117; Hosteller, K. Y., Carson, D. A. and Richman, D. D. (1991); “Phosphatidylazidothymidine: mechanism of antiretroviral action in CEM cells.” J. Biol Chem. 266, 11714 11717; Hosteller, K. Y., Korba, B. Sridhar, C., Gardener, M. (1994a) “Antiviral activity of phosphatidyl-dideoxycytidine in hepatitis B-infected cells and enhanced hepatic uptake in mice.” Antiviral Res. 24, 59 67; Hosteller, K. Y., Richman, D. D., Sridhar. C. N. Felgner, P. L. Felgner, J., Ricci, J., Gardener, M. F. Selleseth, D. W. and Ellis, M. N. (1994b) “Phosphatidylazidothymidine and phosphatidyl-ddC: Assessment of uptake in mouse lymphoid tissues and antiviral activities in human immunodeficiency virus-infected cells and in rauscher leukemia virus-infected mice.” Antimicrobial Agents Chemother. 38, 2792 2797; Hunston, R. N., Jones, A. A. McGuigan, C., Walker, R. T., Balzarini, J., and DeClercq, E. (1984) “Synthesis and biological properties of some cyclic phosphotriesters derived from 2′-deoxy-5-fluorouridine.” J. Med. Chem. 27, 440 444; Ji, Y. H., Moog, C., Schmitt, G., Bischoff, P. and Luu, B. (1990); “Monophosphoric acid esters of 7-β-hydroxycholesterol and of pyrimidine nucleoside as potential antitumor agents: synthesis and preliminary evaluation of antitumor activity.” J. Med. Chem. 33 2264 2270; Jones, A. S., McGuigan, C., Walker, R. T., Balzarini, J. and DeClercq, E. (1984) “Synthesis, properties, and biological activity of some nucleoside cyclic phosphoramidates.” J. Chem. Soc. Perkin Trans. I, 1471 1474; Juodka, B. A. and Smart, J. (1974) “Synthesis of diribonucleoside phosph (P.fwdarw.N) amino acid derivatives.” Coll. Czech. Chem. Comm. 39, 363 968; Kataoka, S., Imai, J., Yamaji, N., Kato, M., Saito, M., Kawada, T. and Imai, S. (1989) “Alkylated cAMP derivatives; selective synthesis and biological activities.” Nucleic Acids Res. Sym. Ser. 21, 1 2; Kataoka, S., Uchida, “(cAMP) benzyl and methyl triesters.” Heterocycles 32, 1351 1356; Kinchington, D., Harvey, J. J., O'Connor, T. J., Jones, B. C. N. M., Devine, K. G., Taylor-Robinson D., Jeffries, D. J. and McGuigan, C. (1992) “Comparison of antiviral effects of zidovudine phosphoramidate and phosphorodiamidate derivatives against HIV and ULV in vitro.” Antiviral Chem. Chemother. 3, 107 112; Kodama, K., Morozumi, M., Saithoh, K. I., Kuninaka, H., Yosino, H. and Saneyoshi, M. (1989) “Antitumor activity and pharmacology of 1-β-D-arabinofuranosylcytosine-5′-stearylphosphate; an orally active derivative of 1-β-Darabinofuranosylcytosine.” Jpn. J. Cancer Res. 80, 679 685; Korty, M. and Engels, J. (1979) “The effects of adenosine- and guanosine 3′,5′phosphoric and acid benzyl esters on guinea-pig ventricular myocardium.” Naunyn-Schmiedeberg's Arch. Pharmacol. 310, 103 111; Kumar, A., Goe, P. L., Jones, A. S. Walker, R. T. Balzarini, J. and DeClercq, E. (1990) “Synthesis and biological evaluation of some cyclic phosphoramidate nucleoside derivatives.” J. Med. Chem., 33, 2368 2375; LeBec, C., and Huynh-Dinh, T. (1991) “Synthesis of lipophilic phosphate triester derivatives of 5-fluorouridine an arabinocytidine as anticancer prodrugs.” Tetrahedron Lett. 32, 6553 6556; Lichtenstein, J., Barner, H. D. and Cohen, S. S. (1960) “The metabolism of exogenously supplied nucleotides by Escherichia coli.,” J. Biol. Chem. 235, 457 465; Lucthy, J., Von Daeniken, A., Friederich, J. Manthey, B., Zweifel, J., Schlatter, C. and Benn, M. H. (1981) “Synthesis and toxicological properties of three naturally occurring cyanoepithioalkanes”. Mitt. Geg. Lebensmittelunters. Hyg. 72, 131 133 (Chem. Abstr. 95, 127093); McGigan, C. Tollerfield, S. M. and Riley, P. a. (1989) “Synthesis and biological evaluation of some phosphate triester derivatives of the anti-viral drug Ara.” Nucleic Acids Res. 17, 6065 6075; McGuigan, C., Devine, K. G., O'Connor, T. J., Galpin, S. A., Jeffries, D. J. and Kinchington, D. (1990a) “Synthesis and evaluation of some novel phosphoramidate derivatives of 3′-azido-3′-deoxythymidine (AZT) as anti-HIV compounds.” Antiviral Chem. Chemother. 1 107 113; McGuigan, C., O'Connor, T. J., Nicholls, S. R. Nickson, C. and Kinchington, D. (1990b) “Synthesis and anti-HIV activity of some novel substituted dialkyl phosphate derivatives of AZT and ddCyd.” Antiviral Chem. Chemother. 1, 355 360; McGuigan, C., Nicholls, S. R., O'Connor, T. J., and Kinchington, D. (1990c) “Synthesis of some novel dialkyl phosphate derivative of 3′-modified nucleosides as potential anti-AIDS drugs.” Antiviral Chem. Chemother. 1, 25 33; McGuigan, C., Devin, K. G., O'Connor, T. J., and Kinchington, D. (1991) “Synthesis and anti-HIV activity of some haloalkyl phosphoramidate derivatives of 3′-azido-3′ deoxythylmidine (AZT); potent activity of the trichloroethyl methoxyalaninyl compound.” Antiviral Res. 15, 255 263; McGuigan, C., Pathirana, R. N., Balzarini, J. and DeClercq, E. (1993b) “Intracellular delivery of bioactive AZT nucleotides by aryl phosphate derivatives of AZT.” J. Med. Chem. 36, 1048 1052.

Alkyl hydrogen phosphate derivatives of the anti-HIV agent AZT may be less toxic than the parent nucleoside analogue. Antiviral Chem. Chemother. 5, 271 277; Meyer, R. B., Jr., Shuman, D. A. and Robins, R. K. (1973) “Synthesis of purine nucleoside 3′,5′-cyclic phosphoramidates.” Tetrahedron Lett. 269 272; Nagyvary, J. Gohil, R. N., Kirchner, C. R. and Stevens, J. D. (1973) “Studies on neutral esters of cyclic AMP,” Biochem. Biophys. Res. Commun. 55, 1072 1077; Namane, A. Gouyette, C., Fillion, M. P., Fillion, G. and Huynh-Dinh, T. (1992) “Improved brain delivery of AZT using a glycosyl phosphotriester prodrug.” J. Med. Chem. 35, 3039 3044; Nargeot, J. Nerbonne, J. M. Engels, J. and Leser, H. A. (1983) Natl. Acad. Sci. U.S.A. 80, 2395 2399; Nelson, K. A., Bentrude, W. G. Stser, W. N. and Hutchinson, J. P. (1987) “The question of chair-twist equilibria for the phosphate rings of nucleoside cyclic 3′,5′ monophosphates. ¹HNMR and x-ray crystallographic study of the diastereomers of thymidine phenyl cyclic 3′,5′-monophosphate.” J. Am. Chem. Soc. 109, 4058 4064; Nerbonne, J. M., Richard, S., Nargeot, J. and Lester, H. A. (1984) “New photoactivatable cyclic nucleotides produce intracellular jumps in cyclic AMP and cyclic GMP concentrations.” Nature 301, 74 76; Neumann, J. M., Herv_, M., Debouzy, J. C., Guerra, F. I., Gouyette, C., Dupraz, B. and Huyny-Dinh, T. (1989) “Synthesis and transmembrane transport studies by NMR of a glucosyl phospholipid of thymidine.” J. Am. Chem. Soc. 111, 4270 4277; Ohno, R., Tatsumi, N., Hirano, M., Imai, K. Mizoguchi, H., Nakamura, T., Kosaka, M., Takatuski, K., Yamaya, T., Toyama K., Yoshida, T., Masaoka, T., Hashimoto, S., Ohshima, T., Kimura, I., Yamada, K. and Kimura, J. (1991) “Treatment of myelodysplastic syndromes with orally administered 1-β-D-arabinouranosylcytosine-5′stearylphosphate.” Oncology 48, 451 455. Palomino, E., Kessle, D. and Horwitz, J. P. (1989) “A dihydropyridine carrier system for sustained delivery of 2′,3′ dideoxynucleosides to the brain.” J. Med. Chem. 32, 22 625; Perkins, R. M., Barney, S. Wittrock, R., Clark, P. H., Levin, R. Lambert, D. M., Petteway, S. R., Serafinowska, H. T., Bailey, S. M., Jackson, S., Harnden, M. R. Ashton, R., Sutton, D., Harvey, J. J. and Brown, A. G. (1993) “Activity of BRL47923 and its oral prodrug, SB203657A against a rauscher murine leukemia virus infection in mice.” Antiviral Res. 20 (Suppl. 1). 84; Piantadosi, C., Marasco, C. J., Jr., Norris-Natschke, S. L., Meyer, K. L., Gumus, F., Surles, J. R., Ishaq, K. S., Kucera, L. S. Iyer, N., Wallen, C. A., Piantadosi, S. and Modest, E. J. (1991) “Synthesis and evaluation of novel ether lipid nucleoside conjugates for anti-HIV-1 activity.” J. Med. Chem. 34, 1408 1414; Pompon, A., Lefebvre, I., Imbach, J. L., Kahn, S. and Farquhar, D. (1994). “Decomposition pathways of the mono- and bis(pivaloyloxymethyl) esters of azidothymidine-5′-monophosphate in cell extract and in tissue culture medium; an application of the ‘on-line ISRP-cleaning HPLC technique.” Antiviral Chem Chemother. 5, 91 98; Postemark, T. (1974) “Cyclic AMP and cyclic GMP.” Annu. Rev. Pharmacol. 14, 23 33; Prisbe, E. J., Martin, J. C. M., McGhee, D. P. C., Barker, M. F., Smee, D. F. Duke, A. E., Matthews, T. R. and Verheyden, J. P. J. (1986) “Synthesis and antiherpes virus activity of phosphate an phosphonate derivatives of 9-[(1,3-dihydroxy-2-propoxy)methyl]guanine.” J. Med. Chem. 29, 671 675; Pucch, F., Gosselin, G., Lefebvre, I., Pompon, a., Aubertin, A. M. Dim, and Imbach, J. L. (1993) “Intracellular delivery of nucleoside monophosphate through a reductase-mediated activation process.” Antiviral Res. 22, 155 174; Pugaeva, V. P., Klochkeva, S. I., Mashbits, F. D. and Eizengart, R. S. (1969). “Toxicological assessment and health standard ratings for ethylene sulfide in the industrial atmosphere.” Gig. Trf. Prof. Zabol. 14, 47 48 (Chem. Abstr. 72, 212); Robins, R. K. (1984) “The potential of nucleotide analogs as inhibitors of Retro viruses and tumors.” Pharm. Res. 11 18; Rosowsky, A., Kim. S. H., Ross and J. Wick, M. M. (1982) “Lipophilic 5′-(alkylphosphate) esters of 1-β-D-arabinofuranosylcytosine and its N⁴-acyl and 2,2′-anhydro-3′-O-acyl derivatives as potential prodrugs.” J. Med. Chem. 25, 171 178; Ross, W. (1961) “Increased sensitivity of the walker turnout towards aromatic nitrogen mustards carrying basic side chains following glucose pretreatment.” Biochem. Pharm. 8, 235 240; Ryu, E. K., Ross, R. J. Matsushita, T., MacCoss, M., Hong, C. I. and West, C. R. (1982). “Phospholipid-nucleoside conjugates. 3. Synthesis and preliminary biological evaluation of 1-β-D-arabinofuranosylcytosine 5′diphosphate[−], 2-diacylglycerols.” J. Med. Chem. 25, 1322 1329; Saffhill, R. and Hume, W. J. (1986) “The degradation of 5-iododeoxyuridine and 5-bromoethoxyuridine by serum from different sources and its consequences for the use of these compounds for incorporation into DNA.” Chem. Biol. Interact. 57, 347 355; Saneyoshi, M., Morozumi, M., Kodama, K., Machida, J., Kuninaka, A. and Yoshino, H. (1980) “Synthetic nucleosides and nucleotides. XVI. Synthesis and biological evaluations of a series of 1-β-D-arabinofuranosylcytosine 5′-alky or arylphosphates” Chem Pharm. Bull. 28, 2915 2923; Sastry, J. K., Nehete, P. N., Khan, S., Nowak, B. J., Plunkett, W., Arlinghaus, R. B. and Farquhar, D. (1992) “Membrane-permeable dideoxyuridine 5′-monophosphate analogue inhibits human immunodeficiency virus infection.” Mol. Pharmacol. 41, 441 445; Shaw, J. P., Jones, R. J. Arimilli, M. N., Louie, M. S., Lee, W. A. and Cundy, K. C. (1994) “Oral bioavailability of PMEA from PMEA prodrugs in male Sprague-Dawley rats.” 9th Annual AAPS Meeting. San Diego, Calif. (Abstract). Shuto, S., Ueda, S., Imamura, S., Fukukawa, K. Matsuda, A. and Ueda, T. (1987) “A facile one-step synthesis of 5′ phosphatidiylnucleosides by an enzymatic two-phase reaction.” Tetrahedron Lett. 28, 199 202; Shuto, S. Itoh, H., Ueda, S., Imamura, S., Kukukawa, K., Tsujino, M., Matsuda, A. and Ueda, T. (1988) Pharm. Bull. 36, 209 217. An example of a useful phosphate prodrug group is the S-acyl-2-thioethyl group, also referred to as “SATE”.

### VI. Methods of Treatment

The compositions described herein can be used to treat patients infected with the HIV-1 and HIV-2.

When the treatment involves co-administration of AZT or other thymidine nucleoside antiviral agents and non-thymidine nucleoside antiviral agents that select for the K65R mutation, it is desirable that the patient has not already developed the K65R mutation. Although the AZT portion of the combination therapy will still be effective, the other agent will be less effective, and perhaps no longer effective.

When the treatment involves co-administration of AZT or other thymidine nucleoside antiviral agents and DAPD, it is desirable that the patient has not already developed the K65R mutation or TAMs. That is, if the patient already has TAMs, the AZT portion of the combination therapy will be less effective, and perhaps no longer effective, and if the patient already has already developed the K65R mutation, the DAPD will be less effective, and perhaps no longer effective.

When the treatment involves the co-administration of an adenine, cytosine, thymidine, and guanine nucleoside antiviral agent, as well as the additional antiviral agent(s), ideally the administration is to a patient who has not yet developed any resistance to these antiviral agents or has been off therapy for at least three months. In that case, it may be possible to actually cure an infected patient if the therapy can treat substantially all of the virus, substantially everywhere it resides in the patient. However, even in the case of infection by a resistant virus, the combination therapy should be effective against all known resistant viral strains, because there is at least one agent capable of inhibiting such a virus in this combination therapy.

Those of skill in the art can effectively follow the administration of these therapies, and the development of side effects and/or resistant viral strains, without undue experimentation.

The present invention will be better understood with reference to the following non-limiting examples.

### Example 1

### In Silico Study to Determine Optimal AZT Dosage Ranges

Current first line highly active antiretroviral therapy (HAART) for the treatment of human immunodeficiency virus (HIV-1) infections combines two nucleoside reverse transcriptase inhibitors (NRTI) together with either a protease inhibitor (PI) or non-nucleoside reverse transcriptase inhibitor (NNRTI) (12, 13, 40). These drug combinations have markedly decreased mortality and morbidity from HIV-1 infections in the developed world (7). Existing therapies cannot eradicate HIV-1 infection because of the compartmentalization of the virus and its latent properties (58, 59). Therefore, chronic therapy remains the standard of care for the foreseeable future. Although HAART regimens are selected in part to minimize cross resistance, and thereby delay the emergence of resistant viruses, all regimens eventually fail, due primarily to lack of adherence to strict regimens, delayed toxicities and/or the emergence of drug-resistant HIV-1 strains (48), making it a major imperative to develop regimens that delay, prevent or attenuate the onset of resistance for second line treatments for infected individuals who have already demonstrated mutations. The occurrence of common resistance mutations, including thymidine analog mutations (TAM), K65R and M184V, need to be a continued focus in the rationale design of HIV-1 NRTI drug development (57).

Data from large genotype databases demonstrated an increased incidence of the K65R mutation from 0.8% in 1998 to 3.8% in 2003, presumably as a result of increased use of tenofovir in clinical practice (55). This mutation produces a single amino acid shift from lysine to arginine in the HIV-1 reverse transcriptase gene, which results in moderate resistance against a variety of NRTI, including tenofovir and abacavir (ABC) (65). In vitro selection of K65R accompanied with moderate resistance has also been demonstrated for other non-thymidine NRTI including zalcitabine, didanosine, adefovir and lamivudine (3TC), beta-2′,3′-didehydro-2′,3′-dideoxy-5-fluorocytidine (d4FC), and beta-D-(2R,4R)-1,3-dioxolane guanosine (DXG) (31, 66). An elevated incidence of K65R and early virological failure have been reported in HIV-1 infected individuals treated with HAART regimens that combine tenofovir with two NRTI, ABC and 3TC. In contrast, individuals treated with the thymidine NRTI, zidovudine (AZT), demonstrate a trend towards decreased emergence of the K65R mutation and better outcomes (25, 39, 49). Furthermore, mechanistic studies demonstrate K65R mutants remain susceptible to thymidine NRTI, including AZT and stavudine (d4T) (6, 21, 30, 31, 46). Therefore, AZT has the potential to serve as a “resistance repellent” agent for the K65R mutation, when combined with NRTI that select for the K65R mutation. The addition of AZT may not be warranted if it competes for rate limiting enzyme phosphorylation with other NRTI contained in the HAART regimen. However, the enzyme used for the rate limiting phosphorylation step of AZT differs from those of tenofovir, abacavir and DXG (2, 3, 14, 16-19, 22, 32, 41, 44, 61).

AZT was the first antiretroviral drug tested in the clinic, initially as a monotherapy drug and later as a component of HAART regimens (7, 11, 20) and was approved as a generic formulation in September 2005 by the United States Food and Drug Administration (FDA). Like other NRTI, AZT undergoes three intracellular phosphorylation steps to form the active triphosphate (AZT-TP). AZT-TP inhibits wild-type HIV-1 reverse transcriptase with an IC₅₀ value of about 0.035 μM (52). The single dose plasma pharmacokinetics of AZT have been well described in HIV-1 infected individuals following intravenous and oral administration, and the systemic clearance (Cl) and a volume of distribution (Vₛₛ) for AZT are in the ranges of 1.1-1.5 l/(kg·hr) and 1.3-1.4 l/kg, respectively (1, 14, 26, 67).

AZT treatment is limited by its toxic side effects in bone marrow cells, resulting in a partially dose dependent incidence of anemia and neutropenia (10, 53, 60). The cytotoxicity of AZT correlates with AZT-MP levels (63). Although the approved oral dose of AZT is 300 mg bid, a study by Barry, et al., (5) suggested that thymidylate kinase may be over-saturated at this dose, since a reduced total dose of AZT 100 mg tid produced similar cellular AZT-TP levels with significantly decreased AZT plasma concentrations and intracellular levels of AZT-MP. This result is in agreement with mechanistic studies which demonstrate that the conversion of AZT-MP to AZT-DP is readily saturated (22). Therefore, if extracellular concentrations of AZT exceed a certain value, then AZT-MP will continue to rise without a further increase in AZT-DP and AZT-TP, which mediates the antiviral effect.

The guanosine nucleoside prodrug of DXG, Amdoxovir ((−)-β-D-2,6-diaminopurine dioxolane; AMDX; DAPD) (8, 19), is being developed by RFS Pharma, LLC, primarily for the second line treatment of HIV-1 infections. To date, over 180 individuals have received DAPD in six different Phase 1 and 2 trials conducted under US investigational new drug applications (IND) (27, 38). Possible advantages of DXG include an increased sensitivity to M184V/I strains in vitro and activity against TAM that may have been selected during previous antiretroviral therapy and 69SS double insert (28, 29, 41). DXG is synergistic with several NRTIs including AZT, 3TC, and nevirapine (28). In vitro studies using HIV-1 in culture with MT-2 cells demonstrated a slow onset of resistance to DXG that was associated with mutations at K65R (23, 47, 66). An in vitro study demonstrated that AZT alone selected for a mixture of K70K/R mutations at week 25, and DAPD alone selected for a mixture of K65R and L74V at week 20. However, when DAPD and AZT were incubated in combination, no drug resistant mutations were detected through week 28 (51.). Therefore, co-formulation of AZT with DAPD may be desirable to delay the emergence of drug resistance in HIV-1 infected individuals due to the K65R mutation.

The objectives of this study were to develop a population pharmacokinetic and pharmacodynamic (PK/PD) model that combines population PK parameters and population statistics of cellular enzyme levels in HIV-1 infected individuals to determine whether the findings of Barry, et al. (5) are supported mechanistically and to develop a dosage regimen of AZT for co-formulation with DAPD and other NRTI, that takes into account possible saturation of thymidylate kinase, while reducing toxicity associated with AZT-MP and maintaining efficacy associated with AZT-TP. A study of AZT 600 mg qd resulted in a slower onset and less pronounced viral depletion than the standard 300 mg bid regimen, which could have resulted from a combination of enzyme saturation of cellular phosphorylation at 600 mg dose and the relatively short cellular half-life of AZT-TP. Therefore, the model was utilized to help select a reduced AZT bid dose that may be suitable for co-formulation with drugs that select for the K65R mutation.

Materials and Methods:

Population Pharmacokinetics of AZT:

The disposition of AZT was assumed to follow the 2-compartment open population pharmacokinetic model of Zhou, et al. (67). The study did not model absorption profiles, due to a lack of data in the absorption phase, and assumed pseudo-zero order absorption kinetics in which fast absorbers (41.7% of individuals) absorbed AZT over 0.25 hr, while slow absorbers (58.3%) absorbed AZT over 1.57 hr. Population characteristics and pharmacokinetic parameters are summarized in Table 1. The equations utilized to generate the actual 2-compartment parameters from population variables in Table 1 were obtained from Zhou, et al. (67). Briefly, volume of distribution at steady-state (Vₛₛ) (L)=464+9.83×(body weight−70)×e(^{Cl}^{_{—}}^{Eta0×Vss}^{_{—}}^{Eta0/Cl}^{_{—}}^{Eta0)} (Eq. 1), where Cl_Eta0 and Vₛₛ_{_{—}}Eta0 are the variances of the log-transformed values of systemic clearance and Vₛₛ, respectively. The term Cl_Eta0×Vₛₛ_{_{—}}Eta0/Cl_Eta0, represents the ratio of variances of natural log-transformed Cl and Vₛₛ, respectively. Cl (1/hr)=127+0.93×(body weight−70)×e^{(Cl}^{_{—}}^{Eta0)}, if age <30 years old, otherwise, Cl (1/hr)=127+0.93×(body weight−70)+6.52×(age−25)×e^{(Cl}^{_{—}}^{Eta0)}) (Eq. 2). Volume of the central compartment (V_{c})=0.374×Vₛₛ (Eq. 3), and the volume of the peripheral tissue compartment (V₂)=Vₛₛ−V_{c} (Eq. 4). The equation used to model AZT concentrations in the plasma during the apparent infusion was: Cₚ=[(D/TI.F)/V₁(K₂₁−α)(α−β)/α](e^{-α.t}−1)+[(D.F/TI)/V₁(K₂₁−β)(α−β)/β]e^{-βt}−1), which becomes Cₚ=[(D.F/TI)/V₁(K₂₁−α)(α−β)/α](e^{-α.t}−e^{-α.(t-TI)})]+[(D.F/TI)/V₁(K₂₁−β)(α−β)/β](e^{-βt}+e^{-β(t-TI)}) after the infusion (Eq. 5) (64). In these equations D is the dose of AZT administered, F is the fraction of AZT absorbed. F is not directly known, since intravenous doses were not available, but is indirectly incorporated in the parameters V₁/F and Cl/F. TI is the apparent duration of “infusion” of AZT in the plasma, β and α are the rate constants of the terminal and next to last exponential decay rates in the plasma, and K₁₂ and K₂₁ are the first-order rate constants describing partitioning of AZT into and out of compartment 2 from compartment 1.

Cytoplasmic Accumulation of AZT and AZT Nucleotides in Activated Peripheral Blood Mononuclear (PBM) Cells In Vivo:

Plasma and cytosolic concentrations of AZT were assumed to achieve rapid equilibration due to the action of equilibrative nucleoside transporters present on the cell membranes of lymphocytes and are described by Eq. 5 (4, 50).

The initial intracellular phosphorylation step of AZT is catalyzed by thymidine kinase (TK). The most likely enzyme for monophosphorylation to AZT-MP is TK₁, which is located primarily in the cytosol of cells in S-phase. However, mitochondrial kinase TK₂ has also been shown to activate AZT in cultured monocytes/macrophages that do not express TK₁, but to a much lesser degree (2, 3, 16, 17, 22, 44). The Kₘ (concentration at 50% of maximal metabolism rate) of AZT versus TK₁ is 0.6 μM (17, 22). The maximal rate of phosphorylation to AZT-MP (V_{max,TK1}) in activated PBM cells was calculated as 0.86 μmol/l per hr, using enzyme kinetic data (35). Since the initial phosphorylation step of AZT to AZT-MP remains approximately linear with dose (5, 22), the V_{max,TK1} was assumed constant between individuals. Thymidylate kinase catalyzes the subsequent phosphorylation to AZT-diphosphate (AZT-DP) and is rate limiting with Kₘ=12 μM versus AZT-MP and V_{max,AZT-MP}=0.3% of the Vₘₐₓ versus thymidine-MP (36). The low Vₘₐₓ of AZT-MP is related to steric hindrance in the binding of AZT-MP to thymidylate kinase (37). The maximal rates of phosphorylation to AZT-MP and AZT-DP (V_{max,AZT-MP} and V_{max,ThymK}, respectively) in activated PBM cells were calculated using previously published enzyme kinetics measurements from a cohort of HIV-1 infected individuals who were not previously treated with AZT (36). The distribution of V_{max,ThymK} followed a log-linear distribution with a median value of 0.13 μmol/l per hr and a SD of log-transformed values of 0.89. Vₘₐₓ values of pmol dTDP from Jacobsson, et al., (36) were converted to μmol AZT-DP/hr taking into account the proportion of activated CD4⁺ PBM cells, since TK₁ is cell cycle dependent and AZT is phosphorylated to a much greater extent in activated than in resting cells (24). The calculation assumed a volume of 0.21 μl/10⁶, corresponding to a protein content of 0.12 mg (3, 33). The final phosphorylation step to active AZT-TP is catalyzed by nucleoside diphosphate kinase and is not rate limiting under physiological conditions. The rates of dephosphorylation of AZT-TP to AZT-DP (K_{TP,DP}), AZT-DP to AZT-MP (K_{DP,MP}), and AZT-MP to AZT (K_{MP}) are similar in vivo (0.28 h⁻¹) (62). At equilibrium the concentrations of AZT-DP and TP are similar. Therefore, the conversion rate of AZT-DP to AZT-TP (K_{DP,TP}) was also assumed to be 0.28 h⁻¹. The assumed distributions for the rate constants describing the cellular phosphorylation and dephosphorylation of AZT are contained in Table 2.

The cytoplasmic accumulation of AZT-MP, -DP and -TP were modeled using the following differential equations:

d(AZT-MP)/dt=Cₚ·V_{max,TK1}/(Cₚ+K_{m,TK1})+AZT-DP·K_{DP,MP}−AZT-MP·K_{MP} (Eq. 6).

d(AZT-DP)/dt=AZT-MP·V_{max,Thmk}/(AZT-MP+K_{m,ThmK})+AZT-TP·K_{TP,DP}−AZT-DP·K_{TP,DP} (Eq. 7).

d(AZT-TP)/dt=AZT-DP·K_{DP,TP}−AZT-TP·K_{TP,DP} (Eq. 8),
where d(AZT-MP)/dt, d(AZT-DP)/dt and d(AZT-TP)/dt=rates of change in cellular AZT-MP, -DP and -TP, respectively.

Computer Simulations:

Monte Carlo population pharmacokinetic and virus dynamic simulations were conducted using Trial Simulator™ version 2.1.2, 2001 (Pharsight Corp., Mountain View, Calif.), which utilizes a 5^{th} order runga-cutta algorithm for numerical integration. This program allows customized differential equations, together with probability distributions of each parameter in the equations to be entered. The pharmacokinetic profile of each theoretical individual was built by randomly selecting each individual's covariate (age, body weight) and PK parameter (e.g. Vₛₛ, Cl₂₁, fast or slow absorber, etc.) and cellular phosphorylation constants (e.g. Vₘₐₓ and Kₘ values for thymidine and thymidylate kinases and decline rate constants for AZT-MP, -DP and -TP), from the distribution summaries contained in Tables 1 and 2, respectively. The parameters of each individual were then used to simulate the plasma concentration versus time profile of AZT for 200 and 300 mg bid doses. The plasma concentration versus time profile for AZT was then used to drive the system of differential equations modeling the accumulation of AZT-MP, -DP and -TP versus time for each dosage regimen. The next individual was then simulated for a total of 3,000 individuals. The final simulated results were analyzed using routines in the S-Plus computer program (version 6.0 Professional, Insightful Corp., Seattle, Wash., 1988) embedded in Trial Simulator™ software. Means and standard deviations versus time were obtained summarizing AZT plasma concentrations and cytoplasmic levels of AZT-MP and AZT-TP. Three simulations of 3,000 individuals each were performed to assess reproducibility of the output.

Results

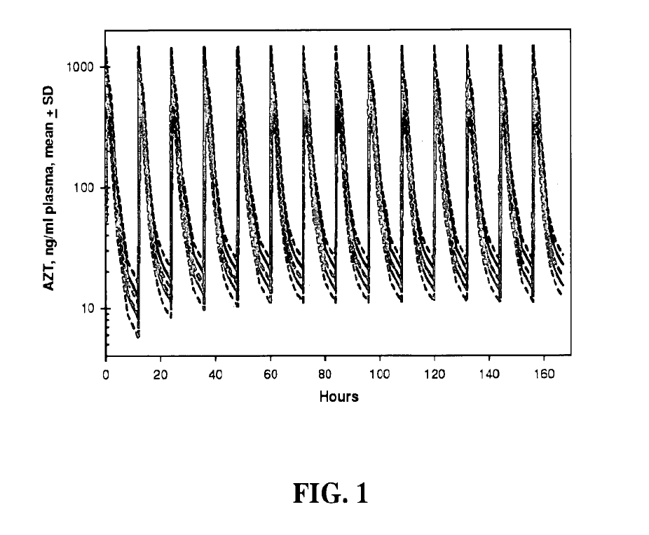

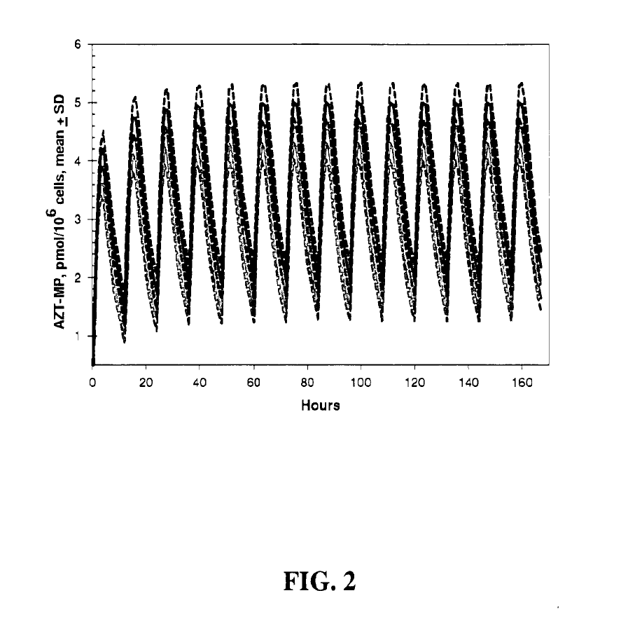

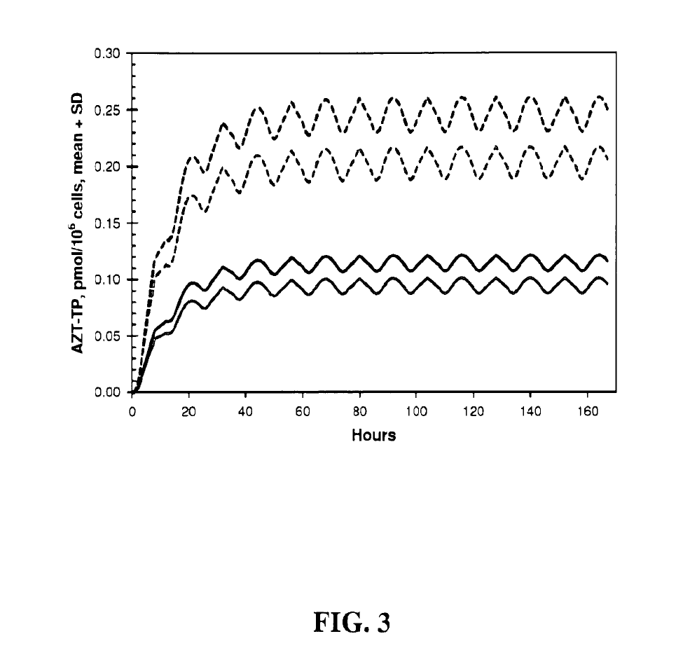

Simulated 200 mg bid dose predicted that plasma concentrations increased linearly with dose (FIG. 1). Similarly cytoplasmic concentration of AZT-MP in activated PBM cells were predicted to increase linearly with dose (FIG. 2). However, the ranges of AZT-TP (mean±SD) (FIG. 3) overlapped considerably for AZT 200 and 300 mg bid, in agreement with super-saturation of AZT phosphorylation at doses close to AZT 200 mg bid. (mean±SD curves for AZT-TP are not shown, since values <0).

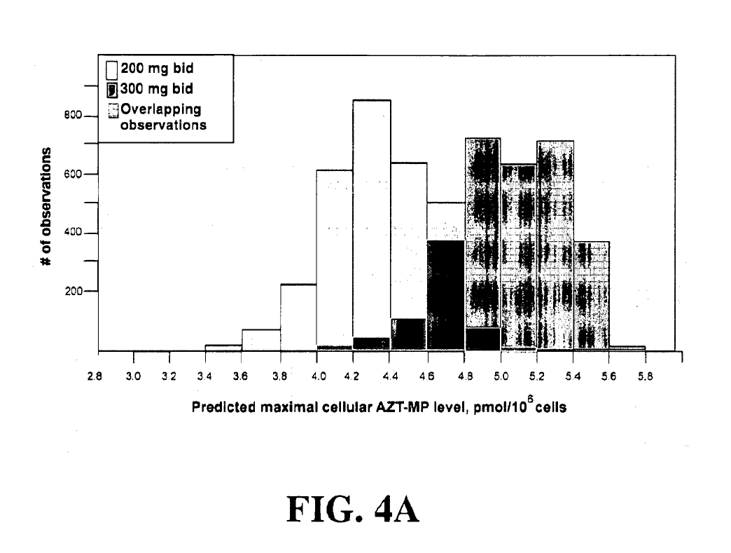

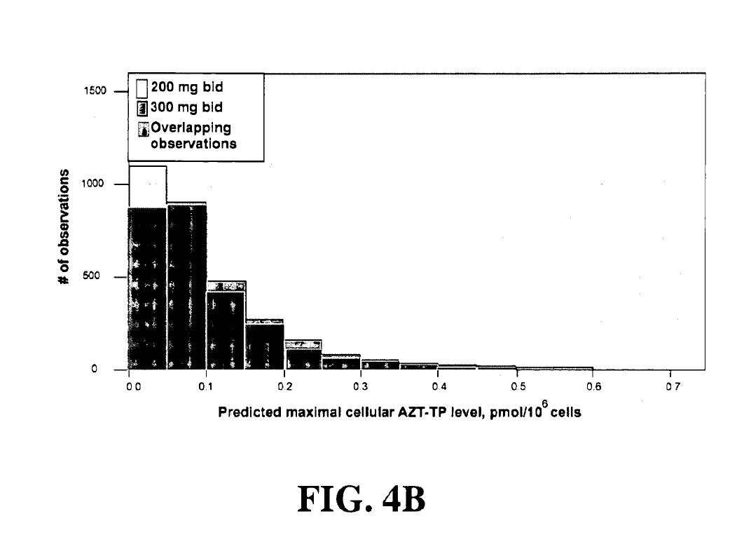

Histograms from three separate simulations of 3,000 individuals each were compiled to compare maximal cytoplasmic concentrations of AZT-MP and AZT-TP. Representative histograms for maximal AZT-MP and AZT-TP are shown in FIG. 4A and FIG. 4B, respectively. There was considerable overlap between the AZT-TP histograms following 200 versus 300 mg bid (>91%, mean±SD of 3 separate simulations: 91.78%±0.25%), while a low degree of overlap was observed between the AZT-MP histograms at the same doses (<23%, mean±SD of 3 separate simulations: 21.93%±0.52%)

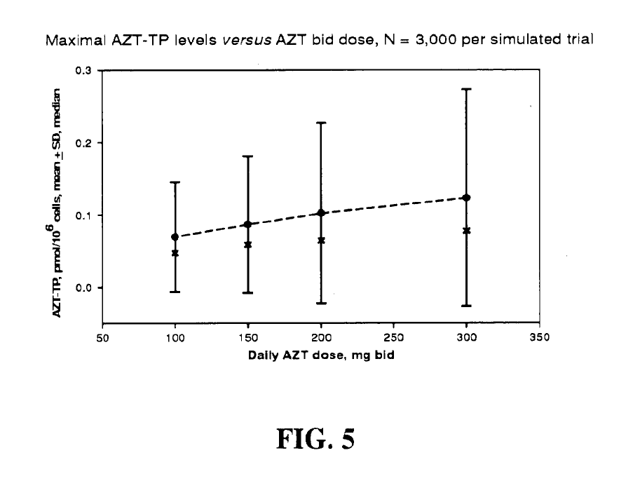

A comparison of maximal AZT-TP levels predicted by simulations with AZT 100, 150, 200 and 300 mg bid (FIG. 5) indicated a decrease in the slope of the dose-response curve between 200 and 300 mg bid doses compared with 100 mg to 200 mg bid, indicative of saturation of thymidylate kinase by the AZT-MP substrate. The relative positions of the means () and medians (x) are indicative of the skewed distributions of the maximal values. Analysis of the data for the 100 mg bid dose suggested that the dose would not be adequate since >50% of observations (N=3,000 simulated individuals) had maximal AZT-TP cytoplasmic concentrations <0.043 pmol/10⁶ cells (EC₅₀).

Discussion:

Pharmacokinetic and pharmacodynamic model simulations are useful tools for consolidating all available drug information in a usable form and are gaining favor in the pharmaceutical industry to design clinical trials, since they allow detailed analyses of dosage regimens in silico before the actual studies are conducted (9, 15, 42, 43, 45). Rosario, et al., in 2006, utilized clinical trial simulations to streamline the phase 2a development of the CCR5 receptor blocking agent maraviroc (54). The objective of the present model was to incorporate the previously reported population pharmacokinetic parameters together with mean and variance estimates of the cellular enzyme kinetics of AZT metabolism of HIV-1 infected individuals, who were not previously treated with AZT, to predict the accumulation of AZT nucleotides in activated CD4⁺ lymphocytes of 3,000 theoretical individuals versus dose regimen. CD4⁺ lymphocytes are the dominant substrates for HIV-1 infection and could be a significant site for the selection of the K65R mutant virus. Furthermore, prediction of AZT-TP levels in activated CD4⁺ PBM cells may be useful for later incorporation into a virus PK-PD model that relates virus depletion profiles versus time and dose of AZT (34). It was desirable to make use of all known drug metabolism factors in the model. However, in silico predictions depend on the model structure together with parameter estimates and their associated variance structures. The parameter distributions were from different studies, so that statistical correlations between parameter covariates could not be analyzed. Therefore, the overall variance in predicted parameters may be overestimated. It is also possible that errors associated with the variance parameters could compound or neutralize each other.

The predicted plasma concentrations of AZT 300 mg bid agree with reported values (3), suggesting that the population pharmacokinetic simulation was reasonable. Both plasma concentrations of AZT and cytoplasmic concentrations of AZT-MP, associated with toxicity, were predicted to increase linearly with dose in the 200 mg to 300 mg bid dose ranges (FIGS. 1 and 2). There was considerable overlap in the simulated AZT-TP levels, responsible for inhibition of viral RT, between the 200 mg and 300 mg bid doses (FIG. 3). The degree of overlap of maximal predicted cytoplasmic concentrations of AZT-MP (FIG. 4A) and AZT-TP (FIG. 4B) showed <23% overlap in maximal cytoplasmic concentrations of AZT-MP and >91% overlap in AZT-TP levels (FIG. 4B). These in silico findings suggest that the AZT dose could be lowered by one-third, from 300 mg bid to 200 mg bid, to reduce toxicities and still maintain adequate AZT-TP levels. Therefore, AZT 200 mg bid may be the optimal dose for co-formulation, maintaining antiviral efficacy, while producing lower toxicity, in support of the study by Barry, et al., (5). However, clinical studies of AZT phosphorylation in infected individuals typically measure nucleotide levels of AZT in PBM cells and do not include a cell cycle analysis of each individual's PBM cells. The cellular capacities of thymidine kinase 1, responsible for the initial phosphorylation of AZT to AZT-MP, and thymidylate kinase, responsible for conversion to AZT-DP are cell cycle dependent, and stimulated PBM cells have been reported to accumulate between 60 to 150 times higher concentrations of AZT nucleotides than resting cells (36, 62). Furthermore, the proportion of dividing PBM cells varies between individuals (36). Therefore, these simulations may not be a direct measure of the AZT nucleotide levels observed in a population of both activated and non-activated cells.

The cellular triphosphate half-lives of tenofovir, DXG and carbovir (active metabolite of abacavir) are: >60 hr, ˜9.5 hr and 12-24 hr, respectively (57). Therefore tenofovir is administered once a day, while DAPD and abacavir are administered twice daily. The phosphorylation to AZT-TP is saturable at high plasma AZT concentrations, and the cellular half-life of AZT-TP is 3-4 hr. This lends mechanistic support to the observation that a 600 mg once daily AZT regimen produces a slower onset and less pronounced viral depletion than the standard 300 mg twice daily regimen of AZT (56). Therefore, although the addition of AZT to a regimen of tenofovir is beneficial, a co-formulation with AZT would result in a less effective AZT response. However, AZT may be a candidate for co-formulation at an optimal dose with NRTI administered twice daily such as DAPD the prodrug of DXG or abacavir.

Based on these in silico results, an intensive pharmacokinetic Phase 2 clinical study sponsored by RFS Pharma, LLC, in 24 HIV-1-infected subjects receiving AZT 200 or 300 mg bid in combination with DAPD 500 mg bid has been completed. This study will provide information on the antiviral effect of the drugs alone and in combination, as well as explore pharmacological interactions with DAPD. The clinical data generated will be important for positioning DAPD in pivotal Phase 3 studies. Furthermore, the utility of a lower dose of AZT without its associated toxicity, especially bone marrow, would be beneficial in future HAART combinations, especially when used with drugs that select for the K65R mutation.

### Tables:

TABLE 1
PK of oral AZT (molecular weight 267.24), with zero order input over
0.25 or 1.57 hr (67).

| Population parameter | Median | Distribution |
|---|---|---|
| Age (years) | 39.2 | normal, % CV = 20, min 20.5, max 60.8 |
| Body weight (kg) | 73.9 | normal, % CV = 22, min 41, max 109 |
| Ln of Var Cl | 0.0 | normal, SD = 0.0703, min −1.06, max 1.06 |
| ¹Vₛₛ_Eta0/Cl_Eta0 | 0.61 | normal, % CV = 46.4, min 0, max 4 |
| ²Cl₂₁ (hr⁻¹) | 27.0 | normal, % CV = 15.2, min 15, max 40 |
| ³fast input time (hr) | 0.25 | fast absorbers = 41.7% of individuals |
| ³slow input time (hr) | 1.57 | slow absorbers = 58.3% of individuals |

^{l}ratio of variance of ln(x) of steady state volume (Vₛₛ) and clearance (Cl) values
²Cl₂₁ represents the inter-compartment clearance value
³Input was approximated as a pseudo-zero-order infusion with varying constant input

TABLE 2
Population enzyme kinetic parameters for cellular metabolism of AZT.

| Parameter | Median | Distribution (references) |
|---|---|---|
| Vₘₐₓ,_{TK1} (μmol/l per hr) | 0.86 | constant (36) |
| K_{m,TK1} (μM) | 0.6 | constant (44) |
| K_{m,ThmK} (μM) | 12.0 | constant (36) |
| ¹V_{max,ThmK} (μmol/l per hr) | 0.13 | Log-normal, SD ln(x) = 0.89 |
| | | (assumed 0.3% of dTMP) (42) |
| K_{MP} = K_{DP,MP},= K_{DP,TP}, = | 0.28 | constant (62) |
| K_{TP,DP} (h⁻¹) | | |
| PBM cells volume | 0.21 | constant (33) |
| (μl/10⁶ cells) | | |
| mg protein per 10⁶ | 0.12 | constant (3) |
| activated PBM cells | | |

¹Mean and SDln(x) values for V_{max,ThmK} were calculated from values measured in PBM cells of nine HIV-1 infected individuals, not previously treated with AZT (36), taking into account the proportion of activated (dividing) CD4⁺ cells. Units of pmol of dTDP formed/min per mg cell protein were converted to μmol AZT-DP/l per hr.

### Example 2

### Synergism Between Amdoxovir and Zidovudine in a Randomized Double Blind Placebo Controlled Study in HIV-Infected Subjects

Background

Amdoxovir (AMDX; DAPD) has been well studied in six trials in close to 200 subjects. In human lymphocytes, zidovudine (AZT) is synergistic with DAPD and prevents selection of K65R and thymidine analog mutations (TAMs). In silico, lower dose AZT may decrease toxicity through the reduction of AZT monophosphate (AZT-MP) accumulation, while maintaining antiviral effect. The study's objective was to determine DAPD's virologic response with and without AZT reduced dose, 200 mg bid, and approved dose, 300 mg bid, in HIV-infected subjects.

Methods

Subjects with HIV RNA viral load (VL)≧5,000 copies/mL were enrolled and randomized 1:1:1 to DAPD 500 mg bid: DAPD 500 mg with AZT 300 mg bid: DAPD 500 mg with AZT 200 mg bid for 10 days. In each arm, subjects were randomized 3:1 to DAPD: placebo. VL was determined daily.

Results

24 subjects [male 54%; white 100%; median age 33 years (range 21-52), median VL 4.5 log₁₀] were enrolled. DAPD/AZT VL decline was more than additive at Day 10, indicating synergy. There was no significant difference in VL decline between DAPD and AZT alone. DAPD/AZT decreased VL variability seen with DAPD. Treatment emergent adverse events were mild to moderate and transient.

| | Mean VL ± SD from baseline (log₁₀ copies/mL) | | |
|---|---|---|---|
| Treatment mg bid (n) | Day 9 | Day 10 | Day 11 |
| Placebo (2) | 0.10* | 0.10* | 0.05* |
| AZT 200 (2) | −0.80 | −0.65 | −0.70 |
| AZT 300 (2) | −0.35 | −0.45 | −0.60 |
| DAPD 500 (6) | −0.83 ± 0.61 | −1.07 ± 0.80 | −1.03 ± 0.59 |
| DAPD + AZT 200 (6) | −1.77 ± 0.27* | −1.97 ± 0.16* | −1.93 ± 0.30* |
| DAPD + AZT 300 (6) | −1.67 ± 0.39* | −1.67 ± 0.21 | −1.75 ± 0.22* |

*p ≦ 0.05 compared with DAPD

Conclusions

DAPD and DAPD/AZT were effective and well tolerated. This proof-of-principle study suggests that long term treatment with DAPD/AZT (200 or 300 mg) should result in synergistic antiviral activity, and further study with AZT 200 mg may demonstrate decreased toxicity.

### Example 3

### Dosage Studies on DAPD/AZT Combination Therapy and Effect on Mean Corpuscle Volume (MCV)

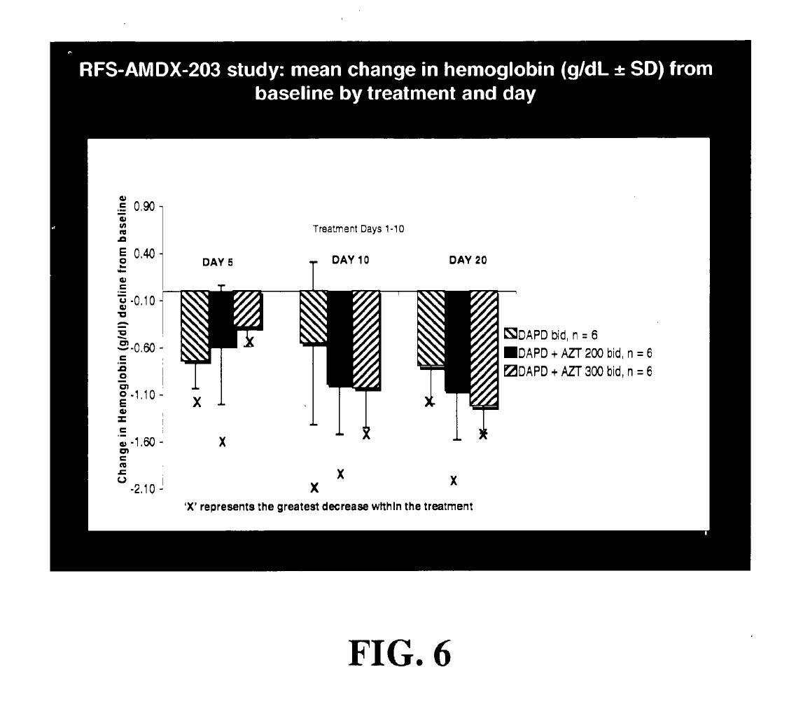

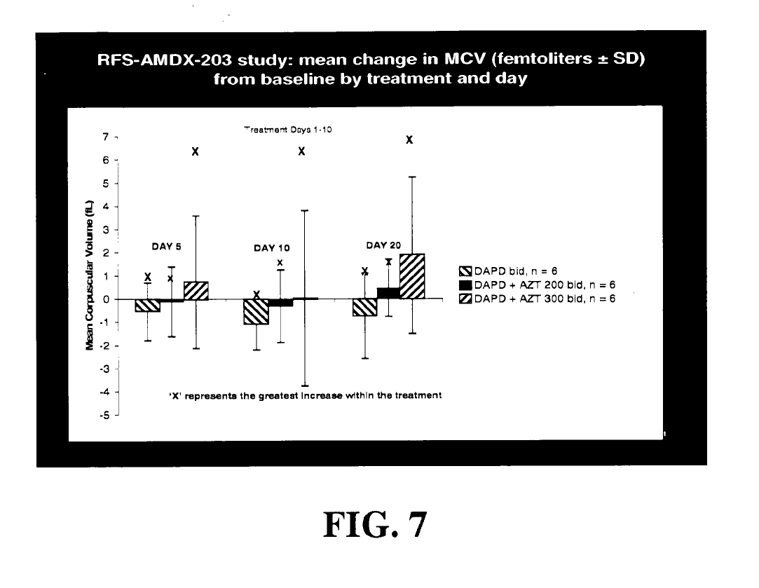

Twenty-four subjects were enrolled [placebo (n=2), AZT 200 mg (n=2), AZT 300 mg (n=2), DAPD 500 mg (n=6), DAPD/AZT 200 mg (n=6), and DAPD/AZT 300 mg (n=6)] and treated for 10 days. Hematological indices including hemoglobin (g/dl) and mean corpuscular volume (MCV, femtoliters) were measured at Screening, Day 1 prior to receiving treatment and Days 5, 10 and 20. One subject who received DAPD/AZT 200 was noted to have a Grade 1 decrease in hemoglobin at Days 10 and 20, and had microcytosis noted at Baseline and at all other sampling points. One subject who received DAPD/AZT 300 had an elevated MCV (97 femtoliters, normal 86±6) noted at Day 20. At Day 20, the trend in decrease in hemoglobin from Baseline was DAPD/AZT 300≧AZT 300≧DAPD/AZT 200>AZT 200>DAPD>placebo (results shown in FIG. 6) and the trend in increase in MCV from Baseline was DAPD/AZT 300>AZT 300>DAPD/AZT 200>AZT 200>placebo>DAPD (results shown in FIG. 7).

### REFERENCES

1. Acosta, E. P., L. M. Page, and C. V. Fletcher. 1996. Clinical pharmacokinetics of zidovudine. An update. Clin Pharmacokinet 30:251-62.
2. Arner, E. S., T. Spasokoukotskaja, and S. Eriksson. 1992. Selective assays for thymidine kinase 1 and 2 and deoxycytidine kinase and their activities in extracts from human cells and tissues. Biochem Biophys Res Commun 188:712-8.
3. Arner, E. S., A. Valentin, and S. Eriksson. 1992. Thymidine and 3′-azido-3′-deoxythymidine metabolism in human peripheral blood lymphocytes and monocyte-derived macrophages. A study of both anabolic and catabolic pathways. J Biol Chem 267:10968-75.
4. Balimane, P. V., and P. J. Sinko. 1999. Involvement of multiple transporters in the oral absorption of nucleoside analogues. Adv Drug Deliv Rev 39:183-209.
5. Barry, M. G., S. H. Khoo, G. J. Veal, P. G. Hoggard, S. E. Gibbons, E. G. Wilkins, O. Williams, A. M. Breckenridge, and D. J. Back. 1996. The effect of zidovudine dose on the formation of intracellular phosphorylated metabolites. Aids 10:1361-7.
6. Bazmi, H. Z., J. L. Hammond, S. C. Cavalcanti, C. K. Chu, R. F. Schinazi, and J. W. Mellors. 2000. In vitro selection of mutations in the human immunodeficiency virus type 1 reverse transcriptase that decrease susceptibility to (−)-beta-D-dioxolane-guanosine and suppress resistance to 3′-azido-3′-deoxythymidine. Antimicrob Agents Chemother 44:1783-8.
7. Carpenter, C. C., D. A. Cooper, M. A. Fischl, J. M. Gatell, B. G. Gazzard, S. M. Hammer, M. S. Hirsch, D. M. Jacobsen, D. A. Katzenstein, J. S. Montaner, D. D. Richman, M. S. Saag, M. Schechter, R. T. Schooley, M. A. Thompson, S. Vella, P. G. Yeni, and P. A. Volberding. 2000. Antiretroviral therapy in adults: updated recommendations of the International AIDS Society-USA Panel. JAMA 283:381-90.
8. Chen, H., R. F. Schinazi, P. Rajagopalan, Z. Gao, C. K. Chu, H. M. McClure, and F. D. Boudinot. 1999. Pharmacokinetics of (−)-beta-D-dioxolane guanine and prodrug (−)-beta-D-2,6-diaminopurine dioxolane in rats and monkeys. AIDS Res Hum Retroviruses 15:1625-30.
9. Chien, J. Y., S. Friedrich, M. A. Heathman, D. P. de Alwis, and V. Sinha. 2005. Pharmacokinetics/Pharmacodynamics and the stages of drug development: role of modeling and simulation. AAPS J 7:E544-59.
10. Collier, A. C., S. Bozzette, R. W. Coombs, D. M. Causey, D. A. Schoenfeld, S. A. Spector, C. B. Pettinelli, G. Davies, D. D. Richman, J. M. Leedom, and et al. 1990. A pilot study of low-dose zidovudine in human immunodeficiency virus infection. N Engl J Med 323:1015-21.
11. Darbyshire, J., M. Foulkes, R. Peto, W. Duncan, A. Babiker, R. Collins, M. Hughes, T. Peto, and A. Walker. 2000. Immediate versus deferred zidovudine (AZT) in asymptomatic or mildly symptomatic HIV infected adults. Cochrane Database Syst Rev:CD002039.
12. de Clercq, E. 1996. Non-nucleoside reverse transcriptase inhibitors (NNRTIs) for the treatment of human immunodeficiency virus type 1 (HIV-1) infections: strategies to overcome drug resistance development. Med Res Rev 16:125-57.
13. Deeks, S. G., M. Smith, M. Holodniy, and J. O. Kahn. 1997. HIV-1 protease inhibitors. A review for clinicians. Jama 277:145-53.
14. Dudley, M. N. 1995. Clinical pharmacokinetics of nucleoside antiretroviral agents. J Infect Dis 171 Suppl 2:S99-112.
15. el-Tahtawy, A. A., T. N. Tozer, F. Harrison, L. Lesko, and R. Williams. 1998. Evaluation of bioequivalence of highly variable drugs using clinical trial simulations. II: Comparison of single and multiple-dose trials using AUC and Cmax. Pharm Res 15:98-104.
16. Eriksson, B. F., C. K. Chu, and R. F. Schinazi. 1989. Phosphorylation of 3′-azido-2′,3′-dideoxyuridine and preferential inhibition of human and simian immunodeficiency virus reverse transcriptases by its 5′-triphosphate. Antimicrob Agents Chemother 33:1729-34.
17. Eriksson, S., B. Kierdaszuk, B. Munch-Petersen, B. Oberg, and N. G. Johansson. 1991. Comparison of the substrate specificities of human thymidine kinase 1 and 2 and deoxycytidine kinase toward antiviral and cytostatic nucleoside analogs. Biochem Biophys Res Commun 176:586-92.
18. Faletto, M. B., W. H. Miller, E. P. Garvey, M. H. St Clair, S. M. Daluge, and S. S. Good. 1997. Unique intracellular activation of the potent anti-human immunodeficiency virus agent 1592U89. Antimicrob Agents Chemother 41:1099-107.
19. Feng, J. Y., W. B. Parker, M. L. Krajewski, D. Deville-Bonne, M. Veron, P. Krishnan, Y. C. Cheng, and K. Borroto-Esoda. 2004. Anabolism of amdoxovir: phosphorylation of dioxolane guanosine and its 5′-phosphates by mammalian phosphotransferases. Biochem Pharmacol 68:1879-88.
20. Fischl, M. A., D. D. Richman, D. M. Causey, M. H. Grieco, Y. Bryson, D. Mildvan, O. L. Laskin, J. E. Groopman, P. A. Volberding, R. T. Schooley, and et al. 1989. Prolonged zidovudine therapy in patients with AIDS and advanced AIDS-related complex. AZT Collaborative Working Group. Jama 262:2405-10.
21. Foli, A., K. M. Sogocio, B. Anderson, M. Kavlick, M. W. Saville, M. A. Wainberg, Z. Gu, J. M. Chemington, H. Mitsuya, and R. Yarchoan. 1996. In vitro selection and molecular characterization of human immunodeficiency virus type 1 with reduced sensitivity to 9-[2-(phosphonomethoxy)ethyl]adenine (PMEA). Antiviral Res 32:91-8.
22. Furman, P. A., J. A. Fyfe, M. H. St Clair, K. Weinhold, J. L. Rideout, G. A. Freeman, S. N. Lehrman, D. P. Bolognesi, S. Broder, H. Mitsuya, and et al. 1986. Phosphorylation of 3′-azido-3′-deoxythymidine and selective interaction of the 5′-triphosphate with human immunodeficiency virus reverse transcriptase. Proc Natl Acad Sci USA 83:8333-7.
23. Furman, P. A., J. Jeffrey, L. L. Kiefer, J. Y. Feng, K. S. Anderson, K. Borroto-Esoda, E. Hill, W. C. Copeland, C. K. Chu, J. P. Sommadossi, I. Liberman, R. F. Schinazi, and G. R. Painter. 2001. Mechanism of action of 1-beta-D-2,6-diaminopurine dioxolane, a prodrug of the human immunodeficiency virus type 1 inhibitor 1-beta-D-dioxolane guanosine. Antimicrob Agents Chemother 45:158-65.
24. Gao, W. Y., R. Agbaria, J. S. Driscoll, and H. Mitsuya. 1994. Divergent anti-human immunodeficiency virus activity and anabolic phosphorylation of 2′,3′-dideoxynucleoside analogs in resting and activated human cells. J Biol Chem 269:12633-8.
25. Gilliam, B. L., M. M. Sajadi, A. Amoroso, C. E. Davis, F. R. Cleghorn, and R. R. Redfield. 2007. Tenofovir and abacavir combination therapy: lessons learned from an urban clinic population. AIDS Patient Care STDS 21:240-6.
26. Gitterman, S. R., G. L. Drusano, M. J. Egorin, and H. C. Standiford. 1990. Population pharmacokinetics of zidovudine. The Veterans Administration Cooperative Studies Group. Clin Pharmacol Ther 48:161-7.
27. Gripshover, B. M., H. Ribaudo, J. Santana, J. G. Gerber, T. B. Campbell, E. Hogg, B. Jarocki, S. M. Hammer, and D. R. Kuritzkes. 2006. Amdoxovir versus placebo with enfuvirtide plus optimized background therapy for HIV-1-infected subjects failing current therapy (AACTG A5118). Antivir Ther 11:619-23.
28. Gu, Z., M. A. Wainberg, N. Nguyen-Ba, L. L′Heureux, J. M. de Muys, T. L. Bowlin, and R. F. Rando. 1999. Mechanism of action and in vitro activity of 1′,3′-dioxolanylpurine nucleoside analogues against sensitive and drug-resistant human immunodeficiency virus type 1 variants. Antimicrob Agents Chemother 43:2376-82.
29. Gu, Z., M. A. Wainberg, P. Nguyen-Ba, L. L′Heureux, J. M. de Muys, and R. F. Rando. 1999. Anti-HIV-1 activities of 1,3-dioxolane guanine and 2,6-diaminopurine dioxolane. Nucleosides Nucleotides 18:891-2.
30. Hammond, J. L., D. L. Koontz, H. Z. Bazmi, J. R. Beadle, S. E. Hostetler, G. D. Kini, K. A. Aldern, D. D. Richman, K. Y. Hostetler, and J. W. Mellors. 2001. Alkylglycerol prodrugs of phosphonoformate are potent in vitro inhibitors of nucleoside-resistant human immunodeficiency virus type 1 and select for resistance mutations that suppress zidovudine resistance. Antimicrob Agents Chemother 45:1621-8.
31. Hammond, J. L., U. M. Parikh, D. L. Koontz, S. Schlueter-Wirtz, C. K. Chu, H. Z. Bazmi, R. F. Schinazi, and J. W. Mellors. 2005. In vitro selection and analysis of human immunodeficiency virus type 1 resistant to derivatives of beta-2′,3′-didehydro-2′,3′-dideoxy-5-fluorocytidine. Antimicrob Agents Chemother 49:3930-2.
32. Hernandez-Santiago, B. I., H. Chen, G. Asif, T. Beltran, S. Mao, S. J. Hurwitz, J. Grier, H. M. McClure, C. K. Chu, D. C. Liotta, and R. F. Schinazi. 2005. Pharmacology and pharmacokinetics of the antiviral agent beta-D-2′,3′-dideoxy-3′-oxa-5-fluorocytidine in cells and rhesus monkeys. Antimicrob Agents Chemother 49:2589-97.
33. Holian, A., C. J. Deutsch, S. K. Holian, R. P. Daniele, and D. F. Wilson. 1979. Lymphocyte response to phytohemagglutinin: intracellular volume and intracellular [K+]. J Cell Physiol 98:137-44.
34. Hurwitz, S. J., and R. F. Schinazi. 2002. Development of a pharmacodynamic model for HIV treatment with nucleoside reverse transcriptase and protease inhibitors. Antiviral Res 56:115-27.
35. Jacobsson, B., S. Britton, Q. He, A. Karlsson, and S. Eriksson. 1995. Decreased thymidine kinase levels in peripheral blood cells from HIV-seropositive individuals: implications for zidovudine metabolism. AIDS Res Hum Retroviruses 11:805-11.
36. Jacobsson, B., S. Britton, Y. Tornevik, and S. Eriksson. 1998. Decrease in thymidylate kinase activity in peripheral blood mononuclear cells from HIV-infected individuals. Biochem Pharmacol 56:389-95.
37. Lavie, A., I. Schlichting, I. R. Vetter, M. Konrad, J. Reinstein, and R. S. Goody. 1997. The bottleneck in AZT activation. Nat Med 3:922-4.
38. Margolis, D., L. Mukherjee, C. Fletcher, E. Hogg, D. Ogata-Arakaki, T. Petersen, D. Rusin, A. Martinez, M. J W, for the ACTG A5165 team. 2007. The use of beta-D-2,6-diaminopurine dioxolane with or without mycophenolate mofetil in drug-resistant HIV infection. AIDS. In press.
39. Martin-Carbonero, L., P. Gil, T. Garcia-Benayas, P. Barreiro, F. Blanco, C. de Mendoza, I. Maida, J. Gonzalez-Lahoz, and V. Soriano. 2006. Rate of virologic failure and selection of drug resistance mutations using different triple nucleos(t)ide analogue combinations in HIV-infected patients. AIDS Res Hum Retroviruses 22:1231-5.
40. Merrill, D. P., M. Moonis, T. C. Chou, and M. S. Hirsch. 1996. Lamivudine or stavudine in two- and three-drug combinations against human immunodeficiency virus type 1 replication in vitro. J Infect Dis 173:355-64.
41. Mewshaw, J. P., F. T. Myrick, D. A. Wakefield, B. J. Hooper, J. L. Harris, B. McCreedy, and K. Borroto-Esoda. 2002. Dioxolane guanosine, the active form of the prodrug diaminopurine dioxolane, is a potent inhibitor of drug-resistant HIV-1 isolates from patients for whom standard nucleoside therapy fails. J Acquir Immune Defic Syndr 29:11-20.
42. Michelson, S., A. Sehgal, and C. Friedrich. 2006. In silico prediction of clinical efficacy. Curr Opin Biotechnol 17:666-70.
43. Miller, R., W. Ewy, B. W. Corrigan, D. Ouellet, D. Hermann, K. G. Kowalski, P. Lockwood, J. R. Koup, S. Donevan, A. El-Kattan, C. S. Li, J. L. Werth, D. E. Feltner, and R. L. Lalonde. 2005. How modeling and simulation have enhanced decision making in new drug development. J Pharmacokinet Pharmacodyn 32:185-97.
44. Munch-Petersen, B., L. Cloos, G. Tyrsted, and S. Eriksson. 1991. Diverging substrate specificity of pure human thymidine kinases 1 and 2 against antiviral dideoxynucleosides. J Biol Chem 266:9032-8.
45. Ogungbenro, K., I. Gueorguieva, O. Majid, G. Graham, and L. Aarons. 2007. Optimal Design for Multiresponse Pharmacokinetic-Pharmacodynamic Models—Dealing with Unbalanced Designs. J Pharmacokinet Pharmacodyn.
46. Parikh, U. M., L. Bacheler, D. Koontz, and J. W. Mellors. 2006. The K65R mutation in human immunodeficiency virus type 1 reverse transcriptase exhibits bidirectional phenotypic antagonism with thymidine analog mutations. J Virol 80:4971-7.
47. Parikh, U. M., D. L. Koontz, C. K. Chu, R. F. Schinazi, and J. W. Mellors. 2005. In vitro activity of structurally diverse nucleoside analogs against human immunodeficiency virus type 1 with the K65R mutation in reverse transcriptase. Antimicrob Agents Chemother 49:1139-44.
48. Pereira, C. F., and J. T. Paridaen. 2004. Anti-HIV drug development—an overview. Curr Pharm Des 10:4005-37.
49. Perez-Elias, M. J., S. Moreno, C. Gutierrez, D. Lopez, V. Abraira, A. Moreno, F. Dronda, J. L. Casado, A. Antela, and M. A. Rodriguez. 2005. High virological failure rate in HIV patients after switching to a regimen with two nucleoside reverse transcriptase inhibitors plus tenofovir. Aids 19:695-8.
50. Plagemann, P. G., R. M. Wohlhueter, and C. Woffendin. 1988. Nucleoside and nucleobase transport in animal cells. Biochim Biophys Acta 947:405-43.
51. Rapp, K. L., M. Ruckstuhl, and R. F. Schinazi. 2007. Presented at the XVI International HIV Drug Resistance Workshop, Barbados, West Indies.
52. Reardon, J. E., and W. H. Miller. 1990. Human immunodeficiency virus reverse transcriptase. Substrate and inhibitor kinetics with thymidine 5′-triphosphate and 3′-azido-3′-deoxythymidine 5′-triphosphate. J Biol Chem 265:20302-7.
53. Richman, D. D., M. A. Fischl, M. H. Grieco, M. S. Gottlieb, P. A. Volberding, O. L. Laskin, J. M. Leedom, J. E. Groopman, D. Mildvan, M. S. Hirsch, and et al. 1987. The toxicity of azidothymidine (AZT) in the treatment of patients with AIDS and AIDS-related complex. A double-blind, placebo-controlled trial. N Engl J Med 317:192-7.
54. Rosario, M. C., B. Poland, J. Sullivan, M. Westby, and E. van der Ryst. 2006. A pharmacokinetic-pharmacodynamic model to optimize the phase IIa development program of maraviroc. J Acquir Immune Defic Syndr 42:183-91.
55. Ruane, P. J., and A. D. Luber. 2004. K65R-associated virologic failure in HIV-infected patients receiving tenofovir-containing triple nucleoside/nucleotide reverse transcriptase inhibitor regimens. MedGenMed 6:31.
56. Ruane, P. J., G. J. Richmond, E. DeJesus, C. E. Hill-Zabala, S. C. Danehower, Q. Liao, J. Johnson, and M. S. Shaefer. 2004. Pharmacodynamic effects of zidovudine 600 mg once/day versus 300 mg twice/day in therapy-naive patients infected with human immunodeficiency virus. Pharmacotherapy 24:307-12.
57. Schinazi, R. F., B. I. Hernandez-Santiago, and S. J. Hurwitz. 2006. Pharmacology of current and promising nucleosides for the treatment of human immunodeficiency viruses. Antiviral Res 71:322-34.
58. Siliciano, J. D., and R. F. Siliciano. 2004. A long-term latent reservoir for HIV-1: discovery and clinical implications. J Antimicrob Chemother 54:6-9.
59. Siliciano, R. F. 2005. Scientific rationale for antiretroviral therapy in 2005: viral reservoirs and resistance evolution. Top HIV Med 13:96-100.
60. Spiga, M. G., D. A. Weidner, C. Trentesaux, R. D. LeBoeuf, and J. P. Sommadossi. 1999. Inhibition of beta-globin gene expression by 3′-azido-3′-deoxythymidine in human erythroid progenitor cells. Antiviral Res 44:167-77.
61. Thompson, M. A., H. A. Kessler, J. J. Eron, Jr., J. M. Jacobson, N. Adda, G. Shen, J. Zong, J. Harris, C. Moxham, and F. S. Rousseau. 2005. Short-term safety and pharmacodynamics of amdoxovir in HIV-infected patients. Aids 19:1607-15.
62. Tornevik, Y., B. Jacobsson, S. Britton, and S. Eriksson. 1991. Intracellular metabolism of 3′-azidothymidine in isolated human peripheral blood mononuclear cells. AIDS Res Hum Retroviruses 7:751-9.
63. Tornevik, Y., B. Ullman, J. Balzarini, B. Wahren, and S. Eriksson. 1995. Cytotoxicity of 3′-azido-3′-deoxythymidine correlates with 3′-azidothymidine-5′-monophosphate (AZTMP) levels, whereas anti-human immunodeficiency virus (HIV) activity correlates with 3′-azidothymidine-5′-triphosphate (AZTTP) levels in cultured CEM T-lymphoblastoid cells. Biochem Pharmacol 49:829-37.
64. Uccellini, D. A., K. Raymond, and D. J. Morgan. 1986. Influence of intravenous infusion duration on the tissue drug concentration profile. J Pharmacokinet Biopharm 14:323-34.
65. Wainberg, M. A., M. D. Miller, Y. Quan, H. Salomon, A. S. Mulato, P. D. Lamy, N. A. Margot, K. E. Anton, and J. M. Chemington. 1999. In vitro selection and characterization of HIV-1 with reduced susceptibility to PMPA. Antivir Ther 4:87-94.
66. White, K. L., N. A. Margot, T. Wrin, C. J. Petropoulos, M. D. Miller, and L. K. Naeger. 2002. Molecular mechanisms of resistance to human immunodeficiency virus type 1 with reverse transcriptase mutations K65R and K65R+M184V and their effects on enzyme function and viral replication capacity. Antimicrob Agents Chemother 46:3437-46.
67. Zhou, X. J., L. B. Sheiner, R. T. D'Aquila, M. D. Hughes, M. S. Hirsch, M. A. Fischl, V. A. Johnson, M. Myers, and J. P. Sommadossi. 1999. Population pharmacokinetics of nevirapine, zidovudine, and didanosine in human immunodeficiency virus-infected patients. The National Institute of Allergy and Infectious Diseases AIDS Clinical Trials Group Protocol 241 Investigators. Antimicrob Agents Chemother 43:121-8.

While the foregoing specification teaches the principles of the present invention, with examples provided for the purpose of illustration, it will be understood that the practice of the invention encompasses all of the usual variations, adaptations and/or modifications as come within the scope of the following claims and their equivalents. All references cited herein are incorporated by reference in their entirety for all purposes.

1. A combination antiretroviral therapy comprising:
a) zidovudine (AZT) or other thymidine nucleoside antiretroviral agents, and
b) one or more non-thymidine nucleoside antiretroviral agents which select for the K65R mutation,
wherein the zidovudine (AZT) or other thymidine nucleoside antiretroviral agent is administered at a dosage at which it, or a triphosphorylated form of the agent, is effective at inhibiting the HIV, but not at a dosage that exceeds the capacity of the phosphorylating enzymes in the patient treated with the therapy such that the monophosphate form of the agent is accumulated in an amount that results in significant side effects.

2. The combination therapy of claim 1, wherein the dosage of zidovudine (AZT) or other thymidine nucleoside antiretroviral agent is between about 100 and about 250 mg bid.

3. The combination therapy of claim 1, wherein the dosage of zidovudine (AZT) or other thymidine nucleoside antiretroviral agent is between about 200 mg bid.

4. The combination therapy of claim 1, wherein the thymidine nucleoside antiretroviral agent is zidovudine (AZT).

5. The combination therapy of claim 1, wherein the non-thymidine nucleoside antiretroviral agent is selected from the group consisting of tenofovir-DF, APD, DAPD, and abacavir (Ziagen).

6. A method for treating an HIV infection in a human comprising administering, in combination or alternation, an effective amount of a composition of claim 1
to a patient in need of treatment thereof.

7. The method of claim 6, wherein the patient has not developed the K65R resistant strain of HIV.

8. The method of claim 6, wherein the dosage of zidovudine (AZT) or other thymidine nucleoside antiretroviral agent is between about 100 and about 250 mg bid.

9. The method of claim 6, wherein the dosage of zidovudine (AZT) or other thymidine nucleoside antiretroviral agent is between about 200 mg bid.

10. The method of claim 6, wherein the thymidine nucleoside antiretroviral agent is zidovudine (AZT).

11. The method of claim 6, wherein the non-thymidine nucleoside antiretroviral agent is selected from the group consisting of tenofovir-DF, APD, DAPD, and abacavir (Ziagen).

12. A combination antiretroviral therapy comprising:
a) zidovudine (AZT) or other thymidine nucleoside antiretroviral agents, and
b) DAPD or APD, wherein the zidovudine (AZT) or other thymidine nucleoside antiretroviral agent is administered at a dosage at which it, or a triphosphorylated form of the agent, is effective at inhibiting the HIV, but not at a dosage that exceeds the capacity of phosphorylating enzymes in a patient receiving such therapy such that the monophosphate form of the agent is accumulated in an amount that results in significant side effects.

13. (canceled)

14. The combination therapy of claim 12, wherein the dosage of zidovudine (AZT) or other thymidine nucleoside antiretroviral agent is between about 100 and about 250 mg bid.

15. The combination therapy of claim 12, wherein the dosage of zidovudine (AZT) or other thymidine nucleoside antiretroviral agent is between about 200 mg bid.

16. A method for treating an HIV infection in a human comprising administering, in combination or alternation, an effective amount of a composition of claim 12
to a patient in need of treatment thereof.

17. (canceled)

18. The method of claim 16, wherein the dosage of zidovudine (AZT) or other thymidine nucleoside antiretroviral agent is between about 100 and about 250 mg bid.

19. The method of claim 16, wherein the dosage of zidovudine (AZT) or other thymidine nucleoside antiretroviral agent is between about 200 mg bid.

20. A combination antiretroviral therapy comprising:
a) at least one each of an adenine, cytosine, thymidine, and guanine nucleoside antiviral agent, and
b) at least one additional antiviral agent selected from the group consisting of non-nucleoside reverse transcriptase inhibitors (NNRTI), protease inhibitors, fusion inhibitors, entry inhibitors, attachment inhibitors, and integrase inhibitors.

21. The combination therapy of claim 20, wherein the thymidine nucleoside antiretroviral agent is zidovudine (AZT).

22. The combination therapy of claim 20, wherein the thymidine nucleoside antiretroviral agent is DAPD or APD.

23. The combination therapy of claim 20, wherein the nucleoside antiretroviral agents comprise:
a) FTC or 3TC,
b) TDF,
c) DAPD or APD,
d) AZT, and
e) a NNRTI, a proteinase inhibitor, or an integrase inhibitor.

24. The combination therapy of claim 23, wherein the NNRTI is Sustiva, the proteinase inhibitor is Kaletra, or the integrase inhibitor is raltegravir.

25. The combination therapy of claim 23, where AZT is used at a dose of 200 mg BID per day.

26. The combination therapy of claim 20, wherein the thymidine nucleoside antiretroviral agent is administered at a dosage at which it, or a triphosphorylated form of the agent, is effective at inhibiting the HIV, but not at a dosage that exceeds the capacity of phosphorylating enzymes in a patient receiving such therapy such that the monophosphate form of the agent is accumulated in an amount that results in significant side effects.

26. (canceled)

27. The combination therapy of claim 12, wherein the dosage of the thymidine nucleoside antiretroviral agent is between about 200 mg bid.

28. A method for treating an HIV infection in a human comprising administering, in combination or alternation, an effective amount of a composition of claim 20
to a patient in need of treatment thereof.

29. An oral dosage form consisting essentially of around 200 mg of AZT, around 500 mg of DAPD, and pharmaceutically acceptable excipients.

30. The oral dosage form of claim 29, further consisting essentially of around 600 mg of efavirenz, around 200 mg emtricitabine, and around 300 mg tenofovir disoproxil fumarate.
